SEQUENCE LISTING (1 sequence)

SEQ ID NO: 1 - protein, 105 aa, Homo sapiens
AMGTGDLDLLSAFRRTSLAGGGRRISDSHEDTGILFSSLLKKRDSFRTPRDSKLEAPAEEDVWEILRQAPPSEYERIAFGYGVTDLRGMLKRLKGMRRDEKKSTA

(19) United States
(12) Patent Application Publication
(10) Pub. No.: US 2023/0349911 A1
(43) Pub. Date: Nov. 2, 2023

(54) COMPOSITIONS AND METHODS TO QUANTIFY THE BINDING INTERACTIONS OF MYOSIN BINDING-PROTEIN C (MYBP-C)

(71) Applicant: ARIZONA BOARD OF REGENTS ON BEHALF OF THE UNIVERSITY OF ARIZONA, Tucson, AZ (US)

(72) Inventors: Brett Colson, Tucson, AZ (US); Fiona Wong, Tucson, AZ (US)

(21) Appl. No.: 18/310,301

(22) Filed: May 1, 2023

Related U.S. Application Data
(60) Provisional application No. 63/336,552, filed on Apr. 29, 2022.

(51) Int. Cl. G01N 33/58

(52) U.S. Cl. CPC G01N 33/582; G01N 2500/04

(57) ABSTRACT

N-terminal cardiac myosin-binding protein C (cMyBP-C) domains (C0-C2) bind to thick (myosin) and thin (actin) filaments to facilitate contraction and relaxation of the heart. These interactions are regulated by phosphorylation of the M-domain situated between domains C1 and C2. In cardiomyopathies and heart failure, phosphorylation of cMyBP-C is significantly altered. A current challenge is to understand myosin- and actin-C0-C2 interactions in the context of mutations and phosphorylation states. The combinatorial analysis needed is challenging with current low-throughput assays. Described herein are time-resolved fluorescence resonance energy transfer (TR-FRET) high-throughput assays to meet this need.

### CROSS-REFERENCES TO RELATED APPLICATIONS

This application is a non-provisional and claims benefit of U.S. Provisional Application No. 63/336,552 filed Apr. 29, 2022, the specification of which is incorporated herein in its entirety by reference.

### REFERENCE TO A SEQUENCE LISTING

The contents of the electronic sequence listing (name of the file ARIZ_22_09_NP_Sequence_Listing.xml; Size: 2,164 bytes; and Date of Creation: Apr. 27, 2023) is herein incorporated by reference in its entirety.

### STATEMENT REGARDING FEDERALLY SPONSORED RESEARCH OR DEVELOPMENT

This invention was made with government support under Grant Nos. R01 HL141564 and T32 HL007249, awarded by National Institutes of Health. The government has certain rights in the invention.

### FIELD OF THE INVENTION

The present invention relates to protein biosensors for drug discovery, in particular, to a high-throughput assay utilizing time-resolved fluorescence energy transfer (TR-FRET) to detect binding interactions.

### BACKGROUND OF THE INVENTION

Myosin binding-protein C (MyBP-C) is a sarcomeric protein responsible for normal contraction and relaxation of the heart. Phosphorylation of MyBP-C fine-tunes cardiac function by regulating its interactions with myosin and actin. The gene encoding for MyBP-C, MYBPC3, is the most mutated gene in hypertrophic cardiomyopathy (HCM), a heritable cardiac disease affecting one in 300-500 people. HCM is characterized by the hypercontractile phenotype and modified protein interactions from mutations in sarcomeric proteins. Together, myosin and MyBP-C account for >60% of HCM mutations. MyBP-C has been identified as a therapeutic target for cardiomyopathies and heart failure due to its altered phosphorylation status in the diseases and its ability to tune contraction by its phosphorylation level. Therefore, targeting MyBP-C with drugs that mimic phosphorylation levels and/or modulate its binding to myosin or actin is a promising approach to treating cardiomyopathies and heart failure.

The cardiac isoform comprises 11 Ig-Fn-like domains, termed C0-C10. The N-terminus (C0-C2) regulates the contractility of cardiac muscle by binding to thick (myosin) and thin (actin) filament proteins. This binding of N-terminal MyBP-C is regulated by the phosphorylation of the M-domain, situated between the C1 and C2 domains. Phosphorylation occurs through activating the beta-adrenergic signaling pathway to enhance the contraction and relaxation of cardiac muscle. Changes in the phosphorylation-regulated binding of MyBP-C to myosin of the thick filament are directly linked to reduced cardiac function, as hypophosphorylation is seen in patients with HCM mutations. It is widely accepted that the N-terminal MyBP-C domains C0-C2 interact with myosin's subfragment-2 (S2) and regulatory light chain (RLC) subunits. Additional interactions of MyBP-C in the central C3-C7 domains with myosin may also be present but do not appear to be modulated by PKA-mediated phosphorylation of MyBP-C.

Current techniques used to investigate the role of phosphorylation in MyBP-C binding to myosin and/or actin are labor intensive, time consuming, and low throughput. In addition to a limited understanding of HCM, this presents a major barrier in efforts to search for therapeutic drugs to correct altered binding in heart diseases. To resolve this, the present invention features newly developed assays that can be utilized to measure the structural dynamics and the functional binding interactions of MyBP-C with myosin and/or actin and identify small-molecule modulators of this binding in screens when combined with high-throughput fluorescence lifetime plate reader technology.

### BRIEF SUMMARY OF THE INVENTION

It is an objective of the present invention to provide systems, compositions, and methods that allow for quantification of structural changes (e.g., binding) between a myosin binding protein C (MyBP-C) and a contractile protein (e.g., a myosin protein, an actin protein, or a fragment thereof) in a high-throughput manner, as specified in the independent claims. Embodiments of the invention are given in the dependent claims. Embodiments of the present invention can be freely combined with each other if they are not mutually exclusive.

The fluorescent protein biosensor comprises a thiol-reactive fluorescent donor probe placed on human regulatory light chain (RLC) of a myosin protein and an acceptor probe on human MyBP-C protein, each at specific cysteine residue sites. FRET is the transfer of energy from the donor to the acceptor probe when they are in close proximity (˜1-10 nanometer distances). Thus, the distances between binding sites in RLC and MyBP-C and occupancy of binding can be detected. Distance/occupancy changes (measured by TR-FRET) in myosin-RLC and MyBP-C report on protein complex structure that is coupled to contractile function. These robust biophysical methods provide structural/functional insight into cardiac diseases and identify potential new therapies to target myosin-MyBP-C binding in cardiac diseases. Potential drug therapies can be identified as those bioactive compounds in a chemical library screen that increase or decrease FRET as a readout for those compounds that increase or decrease myosin-MyBP-C binding, respectively.

In some embodiments, the present invention features a method of using time-resolved fluorescence energy transfer (TR-FRET) and a fluorescent protein biosensor to quantitate protein binding in solution. The method may comprise labeling (i.e., operably connecting) a portion of a contractile protein (e.g., myosin or actin) with a fluorescent donor probe and labeling (i.e., operably connecting) a myosin binding protein-C (MyBP-C) with a fluorescent acceptor probe to generate a fluorescent protein biosensor suitable for TR-FRET. Alternatively, the method may comprise labeling (i.e., operably connecting) a portion of a myosin protein with a fluorescent acceptor probe and labeling (i.e., operably connecting) a myosin binding protein-C (MyBP-C) with a fluorescent donor probe to generate a fluorescent protein biosensor suitable for TR-FRET. In some embodiments, the method comprises measuring FRET efficiency when structural changes in the fluorescent protein biosensor occur and quantitating protein structural changes using the measured FRET efficiency. The FRET efficiency may refer to the proportion of donor molecules that have transferred excitation state energy to acceptor molecules.

In other embodiments, the present invention may also feature an in vitro method for identifying drug candidates for treating hypertrophic cardiomyopathy and/or heart failure using time-resolved fluorescence energy transfer (TR-FRET) and a fluorescent protein biosensor to quantitate protein binding in a solution. The method may comprise labeling (i.e., operably connecting) a portion of a myosin protein with a fluorescent donor probe and labeling (i.e., operably connecting) a myosin binding protein-C (MyBP-C) with a fluorescent acceptor probe to generate a fluorescent protein biosensor suitable for TR-FRET and contacting (e.g., binding) the fluorescent protein biosensor with a drug candidate. Alternatively, the method may comprise labeling (i.e., operably connecting) a portion of a myosin protein with a fluorescent acceptor probe and labeling (i.e., operably connecting) a myosin binding protein-C (MyBP-C) with a fluorescent donor probe to generate a fluorescent protein biosensor suitable for TR-FRET and contacting (e.g., binding) the fluorescent protein biosensor with a drug candidate. In some embodiments, the method comprises measuring FRET efficiency when structural changes in the fluorescent protein biosensor occur, and quantitating protein structural changes (e.g., binding) using the measured FRET efficiency. FRET efficiency may refer to the proportion of donor molecules that have transferred excitation state energy to acceptor molecules.

One of the unique and inventive technical features of the present invention is the use of a fluorescent protein biosensor protein combined with a Fluorescent Lifetime Plate Reader (FLTPR) instrument to measure Time-Resolved Resonance Energy Transfer (TR-FRET). Without wishing to limit the invention to any theory or mechanism, it is believed that the technical feature of the present invention advantageously provides for the quantification of changes in binding of MyBP-C to myosin or actin in a high-throughput manner. This permits precise (i.e., low coefficient of variance and reproducible) screening of bioactive chemical libraries as well as determining the effects of HCM disease mutations in the myosin-MyBP-C protein complex. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, 1D, 1E, 1F, 1G, and 1H show the organization of the sarcomere and the positioning of the TR-FRET probes on myosin, actin, and cMyBP-C. FIG. 1A shows a cartoon of the sarcomere showing the actin thin filaments, myosin thick filaments, and titin between two Z-discs. The C-zone, the thick filament region containing cMyBP-C molecules (vertical stripes), is indicated. The N-terminal domains (C0-C2) of cMyBP-C are indicated between the thin and thick filaments. FIG. 1B shows the subunits and domains of p-cardiac myosin, two heavy chains (grey), two essential light chains (ELC), and two regulatory light chains (RLC). FMAL labeling of RLC is indicated by the triangle. Myosin subfragments generated by selective proteolysis are indicated: Subfragment-1 (S1), Subfragment-2 (S2), heavy meromyosin (HMM), and light meromyosin (LMM). A ribbon diagram of the crystal structure of RLC (PDB: 5TBY) is shown with the position of amino acid 105 used for FMAL labeling indicated (triangle). FIG. 1C shows an F-actin consisting of G-actin monomers (spheres) with FMAL labeling indicated by triangles. A ribbon diagram of the crystal structure of monomeric actin (PDB: 3HBT) is shown with the position of amino acid 374 used for FMAL labeling indicated. FIG. 1D shows a cartoon of cMyBP-C. Immunoglobulin-like domains (circles) and fibronectin type-III domains (hexagons) are depicted, as are the proline/alanine rich linker (P/A) and the M-domain (M) that contains phosphorylation sites (P) and tri-helix bundle (THD). FIG. 1E shows the N-terminal C0-C2 used herein with acceptor probe sites indicated (triangles). FIG. 1F-1H shows ribbon diagrams of the C0 (PDB: 2K1M), and C1 (PDB: 2V6H) domains and the THB (PDB: 5K6P) with the sites used for TMR labeling indicated (triangles).

FIGS. 2A and 2B show FMAL-labeled RLC exchange onto myosin and ATPase activity. FIG. 2A shows representative SDS-PAGE gel monitoring steps of the Myosin-RLC exchange. Lane 1, Protein molecular weight markers; lane 2, unlabeled RLC; lane 3 FMAL-labeled RLC; lane 4, cardiac myosin; lane 5 mixture of myosin with 5× excess FMAL-labeled RLC; lanes 6&7, washes removing excess RLC; lane 8, final myosin containing FMAL-labeled RLC. The top image is the Coomassie stained gel, and the bottom shows the fluorescence signal of labeled RLC for each lane. Further details are described herein. Myosin heavy chain (MHC), myosin essential light chain (ELC) and RLC are labeled. FIG. 2B shows ATPase activity of 1 μM of myosin without (−) RLC exchange (0.040±0.005) and RLC-exchanged (+) myosin (0.07±0.003) were similar in the absence of actin (left) as was actin-activated ATPase activity (0.167±0.029 for unexchanged myosin and 0.161 0.012 for RLC-exchanged myosin) (right). Data shown as mean SE (N=3-4, n=4-6).

FIGS. 3A, 3B, 3C, 3D, 3E, and 3F show fluorescence waveforms of FMAL-labeled myosin and actin and the same together with TMR-labeled C0-C2. FIG. 3A shows normalized fluorescence waveforms of 1 μM Donor (FMAL)-labeled myosin (D, black) and the same in the presence of 20 μM Acceptor (TMR)-labeled C0-C2 (D-A, purple). The instrument response function (IRF) of water scattering is shown (grey). FIG. 3B shows normalized fluorescence waveforms of FMAL-myosin plus TMR-labeled unphosphorylated (D-A, purple) and PKA-phosphorylated (D-A +PKA, dashed purple) C0-C2. Region of fluorescence lifetime, where T decays to 1/e (˜37%) of peak intensity, is highlighted by a dashed box. FIG. 3C shows a magnified dashed box region in FIG. 3B. FIG. 3D shows normalized fluorescence waveforms of 1 μM FMAL-actin (D, black) and the same in the presence of 10 μM TMR-C0-C2 (D-A, green). FIG. 3E shows normalized fluorescence waveforms of FMAL-actin plus TMR-labeled unphosphorylated (D-A, green) and PKA-phosphorylated (D-A +PKA, dashed green) C0-C2. FIG. 3F shows a magnified dashed box region in FIG. 3E.

FIGS. 4A, 4B, 4C, 4D, 4E, and 4F show Myosin-C0-C2 and actin-C0-C2 TR-FRET binding curves. TR-FRET resulting from the binding of unphosphorylated (solid lines) and phosphorylated (dashed lines) TMR-C0-C2 to FMAL-myosin (FIGS. 4A-4C) and FMAL-actin (FIGS. 4D-4F). FIGS. 4A and 4D show TMR FRET acceptor is on C0, C0-C2^{cys85}. FIGS. 4B and 4E show TMR FRET acceptor is on C1, C0-C2^{cys249}. FIGS. 4C and 4F show TMR FRET acceptor is in the THB C0-C2^{cys330}. EC₅₀, maximal FRET (FRETₘₓ), and adjusted R² are shown in Table 1. Data are provided as mean SE (N=3, n=10-15). These actin-C0-C2 TR-FRET binding curves are compared with cosedimentation curves in FIGS. 7A-7I. Arrows indicate TR-FRET values from 5 μM TMR-C0-C2^{cys249} used in Z′-score calculations (Table 2). Arrowheads in all graphs indicate the concentration (2.5 μM) used for comparison of mutant binding (Table 3).

FIGS. 5A, 5B, and 5C show cMyBP-C C0-C2 organization and M-domain mutants. FIG. 5A shows a cartoon depicting C0-C2. The proline/alanine rich linker (P/A) and the M-domain (M), which contains phosphorylation sites (P) and tri-helix bundle (THD), are indicated. FIG. 5B shows the sequence of M-domain (SEQ ID NO: 1) and locations of mutations (*; R282W, E334K, L349R, and L352P) tested for effects on myosin and actin binding. Serine residues phosphorylated by PKA (#) and the PKA recognition sequences (highlighted) are syndicated, as are the helix residues in the THB (thick underlines). C0-C2 probe site cys330 is highlighted and circled. FIG. 5C shows a ribbon diagram of the THB (PDB: 5K6P) with locations of the E334K, L349R, and L352P mutations (*, red). The TMR acceptor labeling site in the THB is indicated (triangle).

FIG. 6 shows FMAL-RLC displays <2% FRET with TMR-C0-C2. 1 μM of unincorporated 10% labeled RLC was mixed with 0-10 μM TMR-C0-C2^{cys249}, and FRET levels determined. For comparison, FRET is shown for the same RLC exchanged onto myosin (black line). Data are provided as mean SE (N=2, n=9-10).

FIGS. 7A, 7B, 7C, 7D, 7E, 7F, 7G, 7H and 7I show actin cosedimentation binding curves for FMAL-Actin and TMR-C0-C2 compared to FRET binding curves. FIGS. 7A-7C shows cosedimentation curves for 1 μM FMAL-actin binding to 0-10.0 μM unphosphorylated (solid black lines) and phosphorylated (dashed lines) TMR-C0-C2. Values are provided as C0-C2/Actin (left X-axis) mean SE (N=2, n=4-6). FRET data (the same data from FIGS. 4D-4F) is included for comparison (solid red and dashed red lines with triangles). Data for C0-C2 with the TMR FRET acceptor on C0 (FIG. 7A, C0-C2^{cys85}), C1 (FIG. 7B, C0-C2^{cys249}), and the THB (FIG. 7C, C0-C2^{cys330}) is shown. Calculated EC₅₀, Bₘₐₓ, and adjusted R2 values are given in Table 4. Linear correlation plots of FRET and cosedimentation binding, normalized to FRETmax and Bmax values of their respective curves, are shown for unphosphorylated (FIGS. 7D-7F) and phosphorylated (FIGS. 7G-7I) TMR-C0-C2.

FIGS. 8A and 8B show FRET and cosedimentation values at submaximal, 2.5 μM, TMR-C0-C2. FRET resulting from 2.5 μM TMR-C0-C2 unphosphorylated (solid bars) and phosphorylated (cross-hatched) for all 3 labeling positions (see legend in FIG. 6), with actin (FIG. 8A) and myosin (FIG. 8B). Also shown in FIG. 8A are values C0-C2/Actin from cosedimentation curves at the same concentration. Cosedimentation values for wild type C0-C2 (with no mutations to remove or add cysteines) is also shown (WT). Values are provided as mean SE (N=2, n=4-6).

FIGS. 9A, 9B, 9C, 9D, 9E, and 9F show TR-FRET testing of TMR-C0-C2 mutants binding to FMAL-myosin. FRET from 2.5, 5.0, and 10.0 μM unphosphorylated (FIGS. 9A, 9C, and 9E, solid bars) and phosphorylated (FIGS. 9B, 9D, and 9F, hatched bars) for wild type (black) the C0-C2 mutants T59A, R282W, E334K, L349R, and L352P. The TMR FRET acceptor is on C0 (C0-C2^{cys85}, FIGS. 9A and 9B), C1 (C0-C2^{cys249}, FIGS. 9C and 9D) or the THB (C0-C2^{cys330}, FIGS. 9C and 9D). Data are provided as mean SE (N=2-3, n=10-15).

FIGS. 10A, 10B, 10C, 10D, 10E, and 10F show TR-FRET testing of TMR-C0-C2 mutants binding to FMAL-actin. FRET curves (0-10.0 μM) for unphosphorylated (FIGS. 10A, 10C, and 10E, solid lines) and phosphorylated (FIGS. 10B, 10D, and 10F, dashed lines) for wild type (black) the C0-C2 mutants T59A, R282W, E334K, L349R, and L352P; note, the lines at the end of the graph are labeled accordingly. The TMR FRET acceptor is on C0 (C0-C2^{cys85}, FIGS. 10A and 10B), C1 (C0-C2^{cys249}, FIGS. 10C and 10D), or the THB (C0-C2^{cys330}, FIGS. 10C and 10D). Data are provided as mean SE (N=2-3, n=10-15).

FIGS. 11A and 11B show the exchange of FMAL-labeled human RLC^{V105C} into cardiac porcine myosin synthetic thick filaments retain normal ATPase function. Fluorescein-maleimide (FMAL)-labeled human ventricular regulatory light chain (RLC) containing labeling site mutation V105C (FMAL-RLC^{V105C}) was biochemically exchanged for native endogenous RLC in porcine cardiac myosin synthetic thick filaments. FMAL-RLC^{V105C} was labeled and properly incorporated into myosin following exchange without loss of function. FIG. 11A shows a coomassie stain of gel bands show total protein (upper panels of bands), and Fluorescence imaging of gel bands show fluorescently-labeled protein (lowest band panel). Lane 1 is FMAL-RLC^{V105C}. Lane 2 is the starting material of myosin with native light chains. Lane 3 is myosin following exchange with FMAL-RLC^{V105C}. From high to low molecular weight on SDS-PAGE gels: myosin heavy chain (HC), essential light chain (ELC), and RLC. FIG. 11B shows an actin-activated ATPase rate (second⁻¹) of non-exchanged myosin and FMAL-RLC^{V105C}-exchanged myosin without (−) or with (+) actin. ATPase rates between non-exchanged and exchanged myosin were not different for − or + actin conditions with low or high ATPase, respectively.

FIG. 12 shows a TR-FRET-binding curve of cardiac myosin-FMAL-RLC^{V105C} donor and human TMR-C0-C2 acceptor with MyBP-C probes in C0, C1, or M-domain are dependent of PKA-mediated phosphorylation. Time-resolved FRET (TR-FRET)-based binding curve of 1 μM FMAL-myosin and 0-20 μM TMR-C0-C2±PKA. FRET Efficiency (% F.E.=1−(T_{DA}/T_{D})) was done for MyBP-C probe in C0 (TMR-C0-C2^{S85C}), C1 (TMR-C0-C2^{C249}), or M-domain (TMR-C0-C2^{P330C}) without (solid lines) and with PKA treatment (dotted lines) to phosphorylate the M-domain of C0-C2.

FIG. 13 shows the effects of HCM missense mutations in C0-C2 on binding to myosin measured by TR-FRET. TR-FRET-based binding of wild type (WT, Cys249 probe) TMR-C0-C2^{C249} compared to hypertrophic cardiomyopathy (HCM) mutant TMR-C0-C2^{C249}: in C0 (T59A) or C1 domains (R282W, E334K, and L352P). FRET Efficiency (%) measurements were made with 1 μM myosin-FMAL-RLC^{V105C} and 2.5, 5, or 10 μM C0-C2 without or with (#p) PKA treatment. Differences in binding at each concentration of mutant C0-C2 compared to WT demonstrate sensitivity of assay to MyBP-C mutations and phosphorylation.

FIGS. 14A and 14B show a Z′-factor test comparing unphosphorylated and phosphorylated C0-C2 and myosin biosensor intensities (FIG. 14A) and lifetimes (FIG. 14B). FIG. 14A shows the intensities of 1 μM FMAL-Donor labeled myosin with 5 μM TMR-Acceptor labeled C0-C2 biosensor without (square) or with PKA treatment (circle) in ˜25 wells each of a 384-well plate. FIG. 14B shows the lifetimes of the same sample. Horizontal dotted lines indicate 3× S.D. of the mean lifetimes (solid lines). Z′-factor is defined as the difference between 3× S.D. divided by the difference in the mean signal. In this test, the Z′-factor for intensities is −6.68 (i.e., overlapping means and 3× S.D.), indicating a useless assay, and the Z′-factor for lifetime is 0.81 (i.e., well-separated means beyond 3× S.D.), indicating an excellent assay for use in HTS.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “biosensor” refers to an analytical device, used for the detection of a chemical substance that combines a biological component with a physicochemical detector. The sensitive biological element, e.g., tissue, microorganisms, organelles, cell receptors, enzymes, antibodies, nucleic acids, etc., is a biologically derived material or biomimetic component that interacts with, binds with, or recognizes the analyte under study. As used herein, the term “biosensor” may also refer to a labeled-MyBP-C and/or a labeled-contractile protein (e.g., a labeled myosin or a labeled actin protein).

As used herein, the “contractile protein” refers to proteins that mediate the sliding of contractile fibers (e.g., contraction) of cardiac and skeletal muscle, e.g., myosin or actin proteins. In some embodiments, “contractile protein” may be used interchangeably with “sarcomeric proteins.” In some embodiments, contractile proteins may be within the sarcomere and include actin and myosin.

As used herein, the term “fluorescent protein biosensor” refers to a biosensor comprising a protein (e.g., a MyBP-C and/or a myosin or actin) and a fluorescent probe operably connected (i.e., a protein labeled with a fluorescent probe).

As used herein, the term “high throughput” refers to the automation of experiments such that large-scale repetition becomes feasible.

As used herein, time-resolved fluorescence refers to time-resolved fluorescence spectroscopy as an extension of fluorescence spectroscopy. Here, the fluorescence of a sample is monitored as a function of time after excitation by a flash of light. The time resolution can be obtained in a number of ways, depending on the required sensitivity and time resolution. In preferred embodiments, direct waveform recording (DWR) is used. In other embodiments, time-correlated single photon counting (TCSPC) is used. In preferred embodiments, DWR or TCSPC is measured in an instrument with plate reader capabilities.

As used herein, fluorescent energy transfer (FRET) refers to a non-radiative transfer of energy between two nearby light-sensitive molecules (fluorophores). Here, a donor dye in an excited state can transfer a part of its energy to an acceptor molecule which then emits fluorescence at a specific wavelength. The emission from the acceptor can be detected as soon as both dyes are in close proximity. In some embodiments, when the two probes are in close proximity the donor emits less light (i.e., the donor emission is quenched when FRET occurs). In some embodiments, the donor and acceptor fluorescence emissions have different wavelengths that can be distinguished from each other.

As used herein, time-resolved fluorescent energy transfer (TR-FRET) refers to the combination of time-resolved (TR) measurement of fluorescence with fluorescence resonance energy transfer (FRET) technology.

As used herein, FRET-efficiency refers to the number of excited donors transferring energy to acceptors over (or divided by) the number of photons absorbed by the donor. FRET-efficiency may also refer to the proportion of the donor molecules that have transferred excitation state energy to the acceptor molecules, which increases with decreasing intermolecular distance.

Described herein, the present invention features a simple high throughput TR-FRET assay to detect the binding of the N-terminal domain of cMyBP-C (C0-C2) to myosin. When used in parallel with a similar assay detecting C0-C2 binding to actin, this makes possible the combinatorial analysis of multiple C0-C2 mutations in the presence and absence of PKA phosphorylation on myosin and actin binding.

The assays herein, myosin and actin, containing fluorescent donor probes, may be combined in solution, e.g., in a 386-well plate, with C0-C2, containing acceptor probes on the C0, C1, or in the tri-helix bundle of the M-domain. Donor probe lifetime is read in minutes, and the resulting intermolecular TR-FRET reports on binding interactions. The usefulness of the assay is demonstrated by analysis of myosin and actin binding of 5 cMyBP-C mutations (T59A, R282W, E334K, L349R, and L352P), in the context of phosphorylation. Reduction in interactions with myosin due to protein kinase A (PKA) phosphorylation of C0-C2 was much greater than effects with actin. The mutations resulted in altered myosin- and actin-FRET profiles for both unphosphorylated and phosphorylated C0-C2. These results suggest that myosin- and actin-based TR-FRET binding assays are suitable for rapid and accurate determination of binding that can be used for investigating C0-C2 mutations and searching for compounds that affect cMyBP-C interactions with myosin or F-actin for therapeutic discovery.

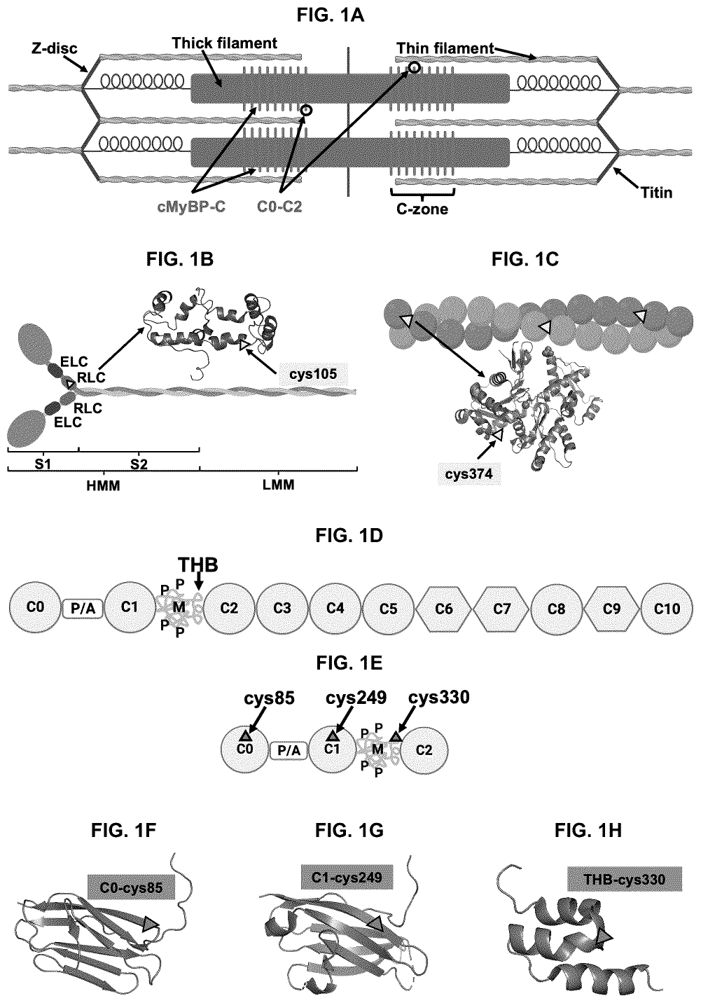

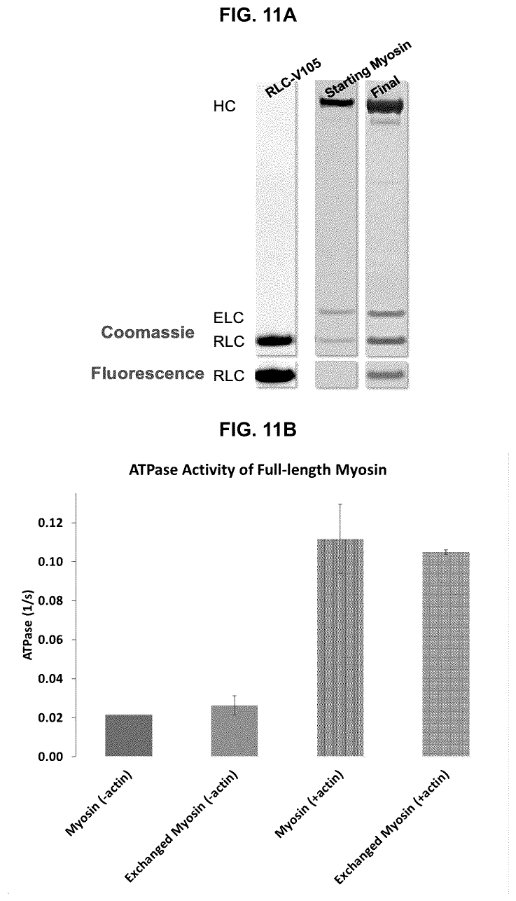

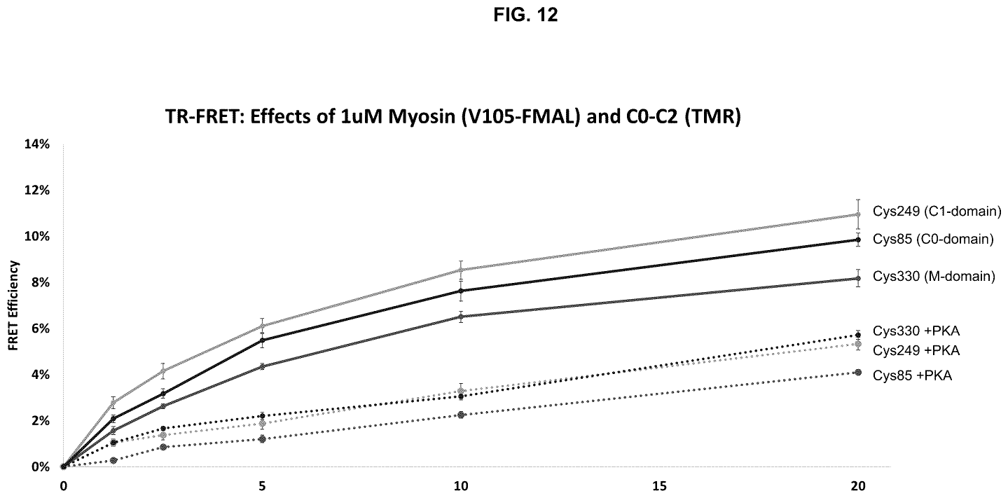

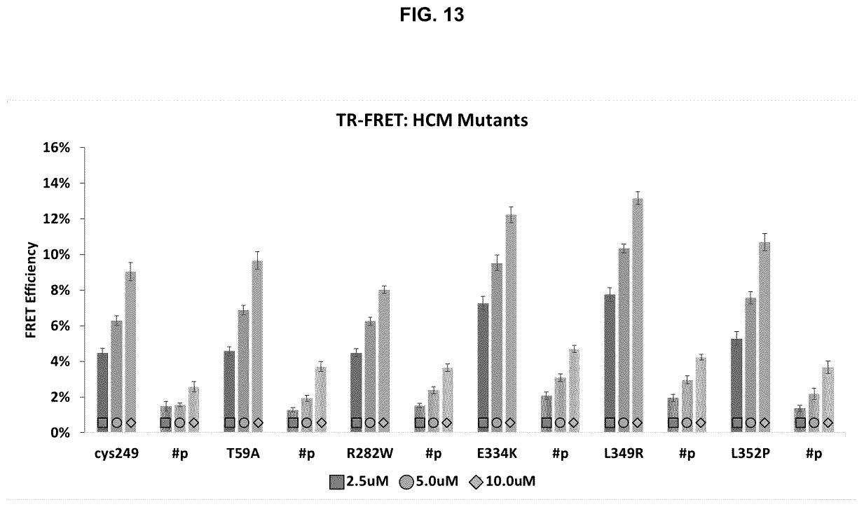

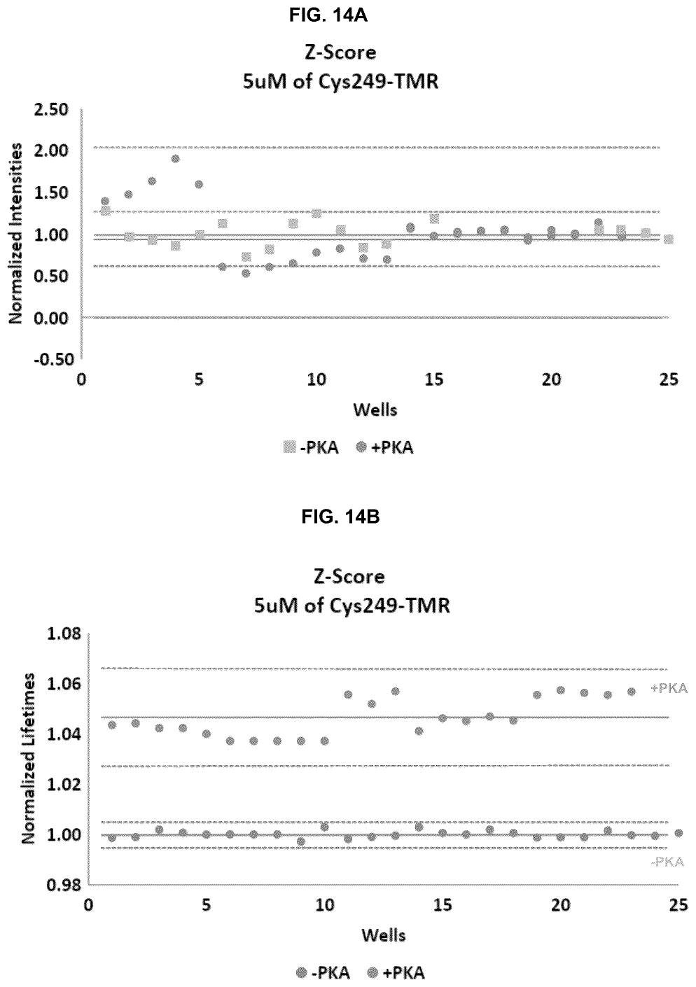

Referring now to FIGS. 1A-14B, the present invention features a fluorescent protein biosensor and method of use of said fluorescent protein biosensor to quantitate structural changes (e.g., protein binding) in solution.

The present invention features a method of using time-resolved fluorescence energy transfer (TR-FRET) and a fluorescent protein biosensor to quantitate structural changes (e.g., protein binding) in solution. The method may comprise a) labeling (i.e., operably connecting) a contractile protein (e.g., an actin protein or a myosin protein) with a first fluorescent probe (e.g., a fluorescent donor probe) and labeling (i.e., operably connecting) a myosin binding protein-C (MyBP-C) with a second fluorescent probe (e.g., a fluorescent acceptor probe) to generate a fluorescent protein biosensor suitable for TR-FRET; b) measuring FRET efficiency when structural changes in the fluorescent protein biosensor occur, wherein FRET efficiency is a proportion of donor molecules that have transferred excitation state energy to acceptor molecules; and c) quantitating protein binding using the measured FRET efficiency.

In some embodiments, the first fluorescent probe comprises a fluorescent donor probe, and the second fluorescent probe comprises a fluorescent acceptor probe. In other embodiments, the first fluorescent probe comprises a fluorescent acceptor probe, and the second fluorescent probe comprises a fluorescent donor probe. The FRET efficiency is the proportion of donor molecules from the fluorescent donor probe that have transferred excitation state energy to acceptor molecules on the fluorescent acceptor probe.

In some embodiments, the method may comprise labeling (i.e., operably connecting) a contractile protein (e.g., a myosin protein or an actin protein) with a fluorescent donor probe and labeling (i.e., operably connecting) a myosin binding protein-C (MyBP-C) with a fluorescent acceptor probe to generate a fluorescent protein biosensor suitable for TR-FRET. Alternatively, the method may comprise labeling (i.e., operably connecting) a contractile protein (e.g., a myosin protein or an actin protein) with a fluorescent acceptor probe and labeling (i.e., operably connecting) a myosin binding protein-C (MyBP-C) with a fluorescent donor probe to generate a fluorescent protein biosensor suitable for TR-FRET. The method may further comprise measuring FRET efficiency when structural changes (e.g., protein binding) in the fluorescent protein biosensor occurs and quantifies protein structural changes using the measured FRET efficiency. The FRET efficiency may refer to the proportion of donor molecules that have transferred excitation state energy to acceptor molecules.

The present invention may also feature an in vitro method for identifying drug candidates for treating hypertrophic cardiomyopathy and/or heart failure using time-resolved fluorescence energy transfer (TR-FRET) and a fluorescent protein biosensor to quantitate structural changes of the fluorescent protein biosensor in a solution. The method may comprise a) labeling (i.e., operably connecting) a contractile protein (e.g., an actin protein or a myosin protein) with a first fluorescent probe (e.g., a fluorescent donor probe) and labeling (i.e., operably connecting) a myosin binding protein-C (MyBP-C) with a second fluorescent probe (e.g., a fluorescent acceptor probe) to generate a fluorescent protein biosensor suitable for TR-FRET; b) contacting the fluorescent protein biosensor with a drug candidate; c) measuring FRET efficiency when structural changes in the fluorescent protein biosensor occur, wherein FRET efficiency is a proportion of donor molecules that have transferred excitation state energy to acceptor molecules; and quantitating protein structural changes using the measured FRET efficiency.

In some embodiments, the method may comprise labeling (i.e., operably connecting) a contractile protein (e.g., an actin protein or a myosin protein) with a fluorescent donor probe and labeling (i.e., operably connecting) a myosin binding protein-C (MyBP-C) with a fluorescent acceptor probe to generate a fluorescent protein biosensor suitable for TR-FRET and contacting (e.g., binding) the fluorescent protein biosensor with a drug candidate. Alternatively, the method may comprise labeling (i.e., operably connecting) a contractile protein (e.g., an actin protein or a myosin protein) with a fluorescent acceptor probe and labeling (i.e., operably connecting) a portion of a myosin binding protein-C (MyBP-C) with a fluorescent donor probe to generate a fluorescent protein biosensor suitable for TR-FRET and contacting (e.g., binding) the fluorescent protein biosensor with a drug candidate. In some embodiments, the method comprises measuring FRET efficiency when structural changes in the fluorescent protein biosensor occur and quantitating protein structural changes (e.g., binding) using the measured FRET efficiency. FRET efficiency may refer to the proportion of donor molecules that have transferred excitation state energy to acceptor molecules.

A variety of drug candidates may be screened in accordance with the in vitro methods described herein (e.g., methods for identifying drug candidates for treating hypertrophic cardiomyopathy and/or heart failure). For example, drugs and/or small molecules that may be used for heart failure, cardiomyopathy and/or muscle disorders.

In some embodiments, the contractile protein (e.g., an actin protein or a myosin protein) and the MyBP-C operably connect or bind in a physiological solution. In some embodiments, the structural changes in the fluorescent protein biosensor comprise an increase or a decrease in the binding of the MyBP-C to the contractile protein (e.g., an actin protein or a myosin protein).

In some embodiments, the contractile protein comprises an actin protein or a myosin protein. The myosin protein may comprise a full-length myosin protein or a fragment thereof. In some embodiments, the full-length myosin protein may include synthetic thick filaments, isolated thick filaments, or a combination thereof. In some embodiments, the fragment of the myosin protein comprises a heavy meromyosin (HMM) fragment, a myosin subfragment 1 (S1), a myosin regulatory light chain (RLC), or a myosin subfragment 2 (S2). The present invention is not limited to the aforementioned fragments of the myosin protein.

The actin protein may comprise an actin filament (F-actin), an actin-tropomyosin (actin-Tm), tropomyosin (Tm), a thin filament (actin-Tm-troponin), troponin, or a fragment thereof. The present invention is not limited to the aforementioned actin proteins.

In some embodiments, the contractile protein may comprise one or more point mutations. In some embodiments, the myosin protein or fragment thereof comprises one or more point mutations. In some embodiments, the actin protein or fragment thereof comprises one or more point mutations.

In some embodiments, the one or more point mutations (within the myosin protein or fragment thereof) comprise a V105C mutation, an A6C mutation, a D94C mutation, a G162C mutation, or a combination thereof. In some embodiments, a fluorescent probe (e.g., a first fluorescent probe; e.g., a fluorescent donor probe) is attached at the point mutation, e.g., the fluorescent probe is attached at the V105C mutation, the A6C mutation, the D94C mutation, or the G162C mutation. In certain embodiments, the fluorescent probe (e.g., a first fluorescent probe; e.g., a fluorescent donor probe) is placed at the V105C mutation. In other embodiments, the fluorescent probe (e.g., a first fluorescent probe; e.g., a fluorescent donor probe) is placed on an endogenous amino acid (e.g., an endogenous cysteine) within the myosin protein or fragment thereof.

In some embodiments, the fluorescent probe (e.g., a first fluorescent probe; e.g., a fluorescent donor probe) is placed at the point mutation within the actin protein or fragment thereof. In other embodiments, the first fluorescent probe (e.g., a fluorescent donor probe) is attached to an endogenous amino acid (e.g., an endogenous cysteine) within the actin protein or fragment thereof, e.g., the fluorescent probe is attached at Cys-374.

In some embodiments, the myosin protein may comprise a point mutation at V105, A6, D94, or G162.

In some embodiments, point mutation made to the aforementioned proteins (e.g., actin, myosin, or a fragment thereof) for placement of a fluorescent probe (e.g., a first fluorescent probe; e.g., a fluorescent donor probe) does not alter the normal function of said protein.

In some embodiments, the MyBP-C comprises a full-length MyBP-C (e.g., C0-C10) or a fragment thereof. Fragments of the MyBP-C may comprise a C0-C2 fragment, a C0-C4 fragment, a C3-C7 fragment, or a C0-C7 fragment. The present invention is not limited to the aforementioned fragments of MyBP-C.

In some embodiments, the fluorescent probe (e.g., a second fluorescent probe; e.g., a fluorescent acceptor probe) is attached to a C0 domain, a C1 domain, an M domain, a C2 domain, a C3 domain, a C4 domain, a C5 domain, or a combination thereof of the MyBP-C. The fluorescent probe, e.g., a second fluorescent probe; e.g., a fluorescent acceptor probe) may be specifically attached to a C249 residue or an H225 residue within the C1 domain, a C528 residue within the C3 domain, a C623 residue within the C4 domain, a C719 residue within the C5 domain, or a combination thereof. In other embodiments, the fluorescent probe (e.g., a second fluorescent probe; e.g., a fluorescent acceptor probe) is attached to a C6 domain, a C7 domain, a C8 domain, a C9 domain, a C10 domain, or a combination thereof.

Without wishing to limit the present invention to any theory or mechanism, it is believed that the fluorescent probe (e.g., a second fluorescent probe; e.g., a fluorescent acceptor probe) is attached to the N-terminal half (e.g., C0-C2 or C0-C4) of MyBP-C allows for the detection (e.g., quantification) of structural changes (e.g., binding) in a dynamic and/or phosphorylation sensitive manner between a myosin binding protein C (MyBP-C) and a contractile protein (e.g., an actin protein and/or a myosin protein).

The MyBP-C may comprise one or point mutation. The one or more point mutations (within the MyBP-C) may comprise an H225C mutation in the C1 domain. In certain embodiments, the fluorescent probe (e.g., a fluorescent acceptor probe) is placed at the H225C mutation. Additional mutations may be made to the MyBP-C being used in said methods to eliminate any other Cys residues not being used to attach a fluorescent probe. For example, when utilizing the C0-C2 fragment, a fluorescent probe (e.g., a fluorescent acceptor probe) may be placed at residue C249; therefore, additional mutations at C239, C426, C436, C443 (e.g., C239L, C426T, C436V, C443S) were made.

In other embodiments, a fluorescent probe (e.g., a fluorescent acceptor probe) is attached to a C0 domain of the MyBP-C. The fluorescent probe (e.g., a fluorescent acceptor probe) may be specifically attached to an S85 residue within the C0 domain. In some embodiments, the portion of the MyBP-C protein comprises a point mutation, such as an S85C mutation within the C0 domain of the MyBP-C, such that the fluorescent probe (e.g., a fluorescent acceptor probe) may be placed at the S85C mutation. In further embodiments, a fluorescent probe (e.g., a fluorescent acceptor probe) is attached to an M domain of the MyBP-C. The fluorescent probe (e.g., a fluorescent acceptor probe) may be specifically attached to a P330 residue within the M domain. In some embodiments, the portion of the MyBP-C protein comprises a point mutation, such as a P330C mutation within the M domain of the MyBP-C, such that the fluorescent probe (e.g., a fluorescent acceptor probe) may be placed at the P330C mutation.

In some embodiments, a fluorescent probe (e.g., a fluorescent acceptor probe) is attached to a C3-C7 fragment of MyBP-C. In some embodiments, a fluorescent probe (e.g., a fluorescent acceptor probe) is attached to a C3 domain, or a C4 domain, or a C5 domain of the MyBP-C. In some embodiments, the fluorescent probe (e.g., a fluorescent acceptor probe) may be specifically attached to a C528 residue within the C3 domain. In some embodiments, the fluorescent probe (e.g., a fluorescent acceptor probe) may be specifically attached to a C623 residue within the C4 domain. In some embodiments, the fluorescent probe (e.g., a fluorescent acceptor probe) may be specifically attached to a C719 residue within the C5 domain. In some embodiments, when the attaching a fluorescent probe to a Cys residue within the C3-C7 fragment of MyBP-C, only one Cys is present for labeling, and all other Cys are removed from the sequence (e.g., any Cys residue not used for labeling may be mutated from to a Ser from a Cys).

The point mutations described herein do not cause improper protein folding/solubility that is notably different from the wild-type protein.

In some embodiments, the MyBP-C further comprises one or more genetic modifications. The one or more genetic modifications are mutations that may be associated with a disease. In some embodiments, the mutations comprise E334K, L349R, T59A, R282W, and L352P. In some embodiments, the disease is heart failure or hypertrophic cardiomyopathy (HCM).

In some embodiments, the fluorescent probe is selected from a group consisting of IAEDANS, IAANS, CPM, IANBD, 5-IAF, TMR, ATTO FMAL, Alexa Fluor 488, Alexa Fluor 532, Alexa Fluor 568, TMR, QSY-9, and DDPM. Other fluorescent probes may be used in accordance with the present invention. The aforementioned fluorescent probes may be used as either a first fluorescent probe (e.g., a fluorescent donor probe) or a second fluorescent probe (e.g., a fluorescent acceptor probe), depending on the excitation wavelength and optical filters. One of ordinary skill in the art would understand how to successfully choose a first fluorescent probe (e.g., a fluorescent donor probe) and a corresponding second fluorescent probe (e.g., a fluorescent acceptor probe).

In some embodiments, a first fluorescent probe (e.g., a fluorescent donor probe) comprises IAEDANS, IAANS, CPM, and IANBD. In some embodiments, a second fluorescent probe (e.g., a fluorescent acceptor probe) comprises DDPM.

In some embodiments, the fluorescent probes comprise thiol-reactive dyes containing a maleimide or iodoacetamide for conjugation with a cysteine on a protein. For example, the fluorescent probes are covalently attached by thiol-reactive chemistry between a cysteine on a protein (e.g., a contractile protein or a MyBP-C) and each dye's maleimide or iodoacetamide group. In some embodiments, the fluorescent probes are thiol-reactive dyes. In other embodiments, the fluorescent probes are amine-reactive dyes.

In further embodiments, the fluorescent probes may comprise other chemistry groups (e.g., amine-reactive), affinity tags (e.g., His-tag or Halo-tag) or peptides (e.g., Lifeact) conjugate with a non-cysteine residue (e.g., lysine or N-terminal amine), peptide-binding region on the protein, or genetically-introduced fluorescent protein (e.g., GFP or RFP). In other embodiments, the fluorescent probes comprise a 355-532 nm excitation range. Without wishing to limit the present invention to any theories or mechanisms, it is believed that red-shifted dyes excited towards the 532 nm help reduce interference with compound autofluorescence in screens. The present invention is not limited to thiol-reactive dyes covalently attached to cysteine residues and may also include, for example, amine-reactive dyes covalently attached to an amine group.

In some embodiments, a solution may refer to a biochemical or physiological buffer. For example, 75 mM KCl and 50 mM Tris, 3 mM MgCl₂, pH 7, or buffers with additional or alternative chemicals. In some embodiments, the solution may have a pH of about 6.5 to 7.5. In some embodiments, the solution may comprise nucleotides and/or ATP. In some embodiments, the methods described herein are ATP-free.

In some embodiments, methods described herein show that by attaching a donor FRET probe to myosin and an acceptor probe to C0-C2, C0-C2-myosin binding and differences in that binding due to phosphorylation or mutations in C0-C2 can be detected. In some embodiments, the method comprises mixing the two labeled proteins in a multi-well (386 wells/plate) format in low volumes (e.g., 50 μl per sample) at low concentrations (e.g., 1-2.5 μM) and measuring fluorescence lifetimes in minutes per plate. In addition to being valuable for the examination of multiple variables important in C0-C2-myosin binding, the assays described herein, may be done in a multi-well format, and are well suited for high throughput screens designed to identify compounds that, like phosphorylation and mutation, modulate C0-C2-myosin binding.

### Example

The following is a non-limiting example of the present invention. It is to be understood that said example is not intended to limit the present invention in any way. Equivalents or substitutes are within the scope of the present invention.

Hypertrophic cardiomyopathy (HCM) is a heritable cardiac disease that affects one in 200-500 people. HCM is characterized by a hypercontractile phenotype and modified protein interactions arising from mutations in sarcomeric proteins, resulting in diastolic dysfunction and cardiac remodeling, which can lead to heart failure (HF). The leading cause of HCM is mutations in the gene encoding cardiac myosin binding protein-C (cMyBP-C), MYBPC3, comprising ˜40-60% of all HCM-associated mutations. cMyBP-C is a sarcomeric protein that interacts with both myosin and actin to facilitate normal contraction and relaxation of the heart. Phosphorylation of cMyBP-C fine-tunes cardiac function by regulating its interactions with myosin and actin. While cMyBP-C is highly phosphorylated under healthy conditions, in disease states, including those associated with mutations in other sarcomeric proteins, phosphorylation levels of cMyBP-C are significantly decreased. Understanding how normal and HCM mutant cMyBP-C are affected by phosphorylation-sensitive binding to myosin and actin can provide therapeutic insights for the realization of cMyBP-C as a target for drug compounds to adjust its activities to treat HCM and/or HF.

cMyBP-C is a ˜140 kDa thick filament-associated protein sarcomeric protein comprised of 8 immunoglobulin-like and 3 fibronectin-like domains termed C0-C10 (FIGS. 1A and 1D). The C-terminus (domains C8-C10) is anchored to the thick filament backbone with binding sites to light meromyosin and titin. The N-terminal domains (C0-C2) contain binding sites to myosin and actin. Within the C0-C2 domain, C0 and C1 domains are connected by a proline-alanine-rich linker (P/A linker), and C1 and C2 are joined by the M-domain. NMR experiments suggest that the M-domain has a largely disordered N-terminal portion containing four serine residues that can be phosphorylated: Ser275, Ser284, Ser304, and Ser311 and a more ordered C-terminal subdomain that contains 3 alpha helices arranged in a tri-helix bundle (THB).

The binding of cMyBP-C to actin and myosin is enhanced in the unphosphorylated state. This binding can activate the thin filament through interactions with actin and tropomyosin and also act as a brake to contraction by decreasing activation of the thick filament. During relaxation, cMyBP-C binding can again act as a brake by slowing relaxation through postulated drag mechanisms. Phosphorylation of cMyBP-C mediated by protein kinase A, protein kinase C and Ca²⁺-calmodulin-activated kinase II results in structural changes altered interactions with myosin and actin, and changes in the activating and braking effects.

cMyBP-C phosphorylation is commonly dysregulated and reduced in patients with HCM or HF, resulting in dyssynchronous myosin and actin interactions. Functional studies suggest that phosphorylated cMyBP-C is cardioprotective as mice expressing phosphomimetic charge substitutions at protein kinase A (PKA)-mediated phosphorylation sites display preserved cardiac morphology, enhanced myocardial relaxation, and attenuated age-related cardiac dysfunction. That cMyBP-C phosphorylation is reduced in diseased conditions suggests that HCM and HF are, at least partially, mediated by increased binding of cMyBP-C to actin and/or myosin to levels that result in pathology.

Hundreds of mutations in MYBPC3 (the gene encoding cMyBP-C) are linked to the development of cardiomyopathy and heart failure, but the impact of these mutations on phosphorylation-regulated cMyBP-C binding interactions with myosin and actin remains limited due to the lack of high-throughput binding assays. Described herein are robust high-throughput fluorescence lifetime-based assays to study the dynamic structure of C0-C2 and the binding interactions between C0-C2 and F-actin in unphosphorylated, phosphorylated, and mutant conditions. Screening of 1,280 pharmacologically active compounds with one of these assays resulted in the identification of the first three drugs that bind to cMyBP-C and abolish its interactions with F-actin. These drugs were further characterized for C0-C2-actin binding using a TR-FRET-based C0-C2-actin binding assay. In this TR-FRET assay, a donor fluorescent probe (e.g., Fluorescein-5-maleimide (FMAL)) was attached to actin on cys374 (FIG. 1C), and an acceptor probe (e.g., tetramethylrhodamine (TMR)) was attached to the C1 domain on cys249 in C0-C2 (FIG. 1G). Binding of C0-C2 to actin brought the acceptor probe close enough to the donor probe to result in resonance energy transfer. This was detected as a change in lifetime (the time it takes for the donor intensity to decay from its maximum intensity following excitation, to ˜37% (1/e)). The C0-C2 actin interaction TR-FRET assays were further developed and, for the first time, an assay to accurately measure interactions between cMyBP-C and myosin has been established. For myosin labeling, the donor, FMAL, was placed on the RLC myosin subunit containing the cys105 mutation (FIG. 1B). RLC has no endogenous cysteine residues and the introduction of a cysteine at residue 105 (cys105) provides a convenient site for thiol labeling. This was then exchanged onto purified full-length porcine ventricle myosin. C0-C2 containing a single cysteine in the C0 (cys⁸⁵), C1 (cys²⁴⁹), and tri-helix bundle (THB) of the M (cys³³⁰) domains were used for acceptor (TMR) labeling (FIG. 1E-1H). The FMAL-TMR FRET pair has an R0 of 5.5 nm, meaning that probes separated by up to 8.25 nM (˜1.5× R0) can be reliably detected. Modeling of C0-C2 docked with myosin interacting-heads motif indicates that this is an appropriate distance between RLC of myosin and N-terminal cMyBP-C domains.

Using these TR-FRET assays, unphosphorylated and phosphorylated C0-C2 binding to myosin and actin can now be studied. To further validate these assays, the effects of 5 potential C0-C2 mutations (T59A, R282W, E334K, L349R, and L352P) were examined that are thought to be responsible for HCM or have predictable effects on myosin and actin interactions. Finally, for therapeutic drug discovery, these TR-FRET assays were tested for usefulness in future high-throughput screens designed to identify compounds that modulate C0-C2 actin and myosin binding interactions.

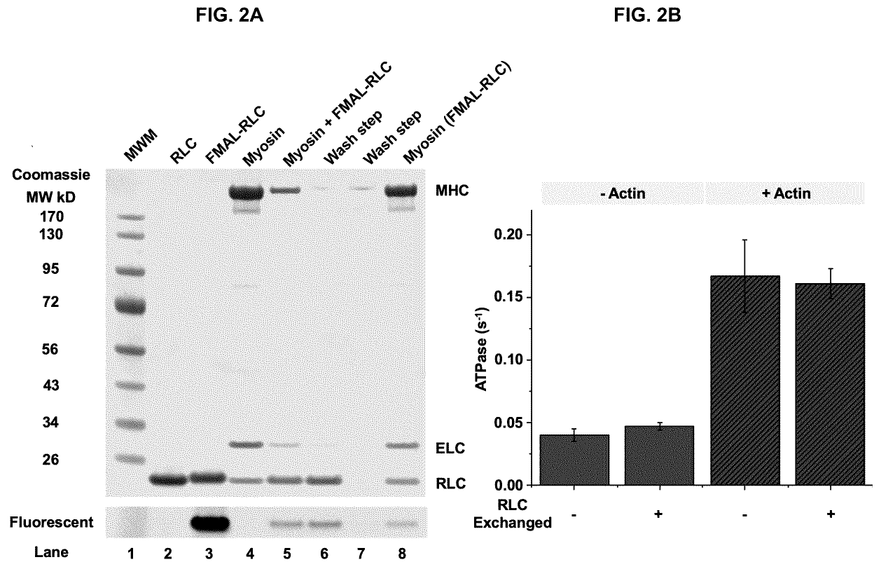

TR-FRET probes for detection of C0-C2 binding to myosin and actin: Site-directed mutagenesis was used to introduce a cysteine in the regulatory light chain (RLC) subunit at residue 105, cys105 (FIG. 1B). To ensure that the fluorescein-5-maleimide (FMAL) on one RLC did not interact with FMAL on a neighboring RLC only 10% labeled RLC was exchanged onto myosin. This was accomplished by using a mixture of FMAL-labeled and unlabeled RLC for exchange. RLC exchange was monitored by analyzing SDS-PAGE gels for protein and fluorescence staining (FIG. 2A). F-actin was labeled at site Cys-374 (FIG. 1C). Labeled and unlabeled G-actin were mixed to achieve 10% labeled monomers in the final F-actin filaments. To monitor C0-C2-myosin binding and C0-C2-actin binding, acceptor probes were inserted at different positions (e.g., C0; cys85, C1; cys249; the THB in the M-domain; cys330) in the cMyBP-C N-terminal C0-C2 (FIG. 1E-1G). C0-C2 contains 5 endogenous cysteines, which were strategically removed to allow for insertions of single cysteines, cys85 (FIG. 1F) and cys330 (FIG. 1H). Cys249 of the C1 domain is an endogenous, surface-accessible thiol, so the remaining 4 cysteines were removed to generate this protein (FIG. 1G).

To test FMAL-RLC exchanged myosin functionality, its steady-state ATPase rates were measured and compared to unexchanged wild-type myosin (WT) (FIG. 2B). The basal and actin-activated myosin ATPase activities of 0.040±0.005 s⁻¹ and 0.167±0.029 s⁻¹ for the RLC-exchanged myosin were comparable to the rates of unexchanged myosin with ATPase activities of 0.047±0.003 s⁻¹ and 0.161±0.012 s⁻¹, respectively. These results demonstrated that the RLC exchanged myosin was functionally similar to unexchanged myosin and retained normal ATPase function.

High-throughput myosin-C0-C2 binding TR-FRET Assay: In the myosin-C0-C2 TR-FRET binding assay, a FRET donor probe is placed on myosin and a FRET acceptor probe is attached to C0-C2. Binding of C0-C2 to myosin brings the acceptor probe in close proximity to the donor probe resulting in FRET, a decrease in the donor probe's lifetime. The donor FRET probe on myosin is localized to the regulatory light chain (RLC) on a single cysteine using a thiol-reactive fluorescent dye. RLC has no cysteines and so a cysteine was introduced at one site Valine-105 (cys¹⁰⁵) for fluorescent labeling. Labeled RLC was exchanged onto full-length myosin (FIG. 2A) with modifications for using cardiac full-length filamentous myosin rather than smooth muscle myosin S1 fragments.

Synthetic thick filaments comprised of full-length myosin were used as this form contains all of the myosin subdomains, such as the regulatory light chain (RLC), heavy meromyosin (HMM), light meromyosin (LMM), subfragment 1 (S1), and subfragment 2 (S2) that together with MyBP-C and titin interactions are responsible for stabilizing the interacting heads motif (IHM).

For the myosin-C0-C2 TR-FRET binding assay, FMAL was used as the donor probe on myosin and TMR as the acceptor probe on C0-C2. The Förster radius (R0, the distance at which 50% FRET occurs) of FMAL and TMR is 5.5 nm. Thus, these FRET assays can detect the binding of C0-C2 to actin and myosin that brings probes within ˜9-10 nm of each other. By testing C0-C2 labeled with TMR for FRET with FMAL labeled myosin or actin in paired experiments using the same labeled C0-C2, the effects of phosphorylation or mutation effects on myosin versus actin binding can be more directly compared.

C0-C2 constructs with single cysteines (for TMR labeling) located in the C0, C1, or THB of the M-domain were tested. This ensured that differences in binding due to phosphorylation or MyBP-C mutations are not due to artifacts of placing the probe in a particular location. It also allowed potential FRET differences to be probed that result from localizing TMR in a particular domain that might be closer or further away from the FMAL in myosin or actin. Probes located across C0-C2 also offer a more nuanced picture of C0-C2 interactions with myosin and actin.

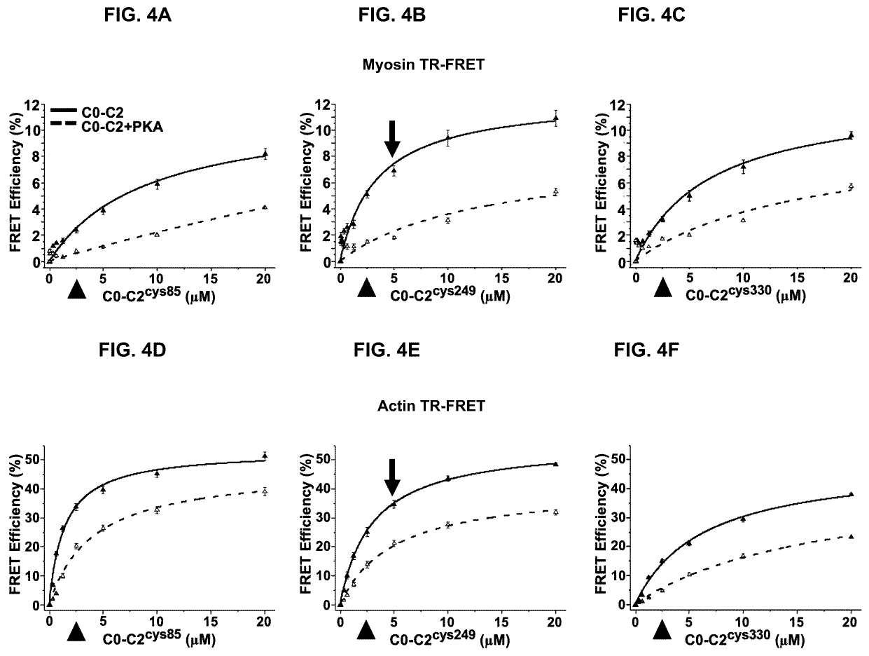

Myosin versus actin binding to C0-C2: Binding curves for TMR-labeled MyBP-C C0-C2 binding to myosin and actin were readily obtained by calculating TR-FRET values from lifetime observations (FIG. 4A-4F). All three C0-C2 constructs (acceptor probes in (C0, C1, and in the THB of the M-domain)) showed significant reductions in FRET between C0-C2 and myosin or actin upon PKA phosphorylation of C0-C2. This is consistent with the decreased C0-C2 binding to myosin and to actin. Reductions in FRET can also be the result of altered modes of binding (e.g., binding that increases the distance or disorder between donor and acceptor probes).

EC₅₀s of C0-C2 binding to myosin and actin are similar, 1.4-9 μM, though the quantitative order of EC₅₀s between TMR placed in C0, C1 and the THB are different. In the myosin-C0-C2 TR-FRET binding assay, the TMR on C1 (C0-C20^{cys249}) displayed the lowest EC₅₀ (3.4±1.1 μM), followed by TMR on the THB (C0-C2^{cys330}) (EC₅₀=6.9±2.5 μM) and lastly TMR on C0 (C0-C2^{cys15}) had the highest EC₅₀ (9.0±2.6 μM). The range of EC₅₀ values (3.4-9.0 μM) for myosin binding are consistent with the 2.8-8.6 μM range of K_{D} values found using cosedimentation and MST and binding assays. For actin TR-FRET assays the order of EC₅₀'s was different, with the lowest EC₅₀ being displayed by TMR on C0 (C0-C2^{cys85}, EC₅₀=1.4±0.2), then C1 (C0-C2^{cys249}, EC₅₀=3.0±0.1), and lastly THB (C0-C2^{cys330}, 6.3±0.7). The range of EC₅₀ values (1.4-6.3 μM) for actin binding are consistent with the 2.5-13.7 μM range of K_{D} values observed in other binding assays.

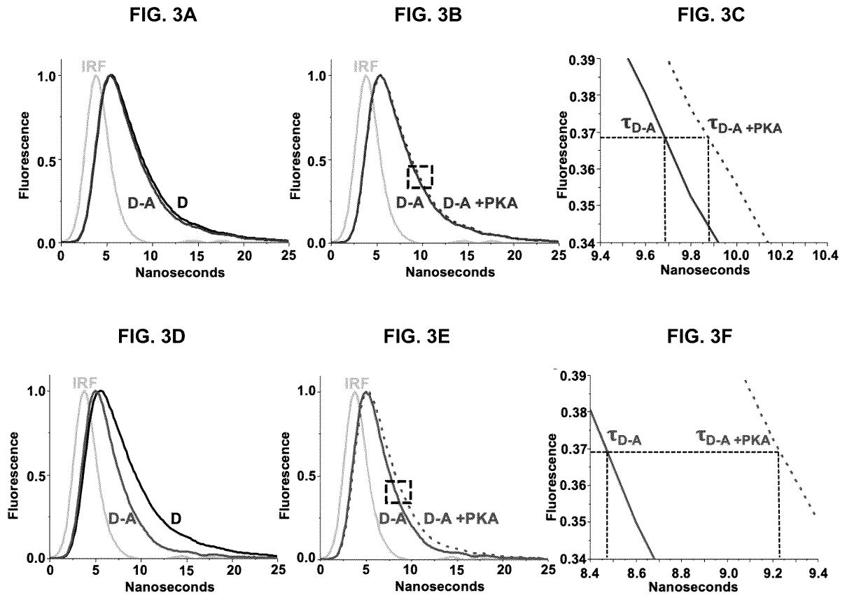

TR-FRET C0-C2-myosin and C0-C2-actin binding curves: Fluorescence waveforms of FMAL-labeled myosin and actin, and the same in the presence of TMR-labeled C0-C2 were analyzed by one-exponential fitting to determine fluorescence lifetime (FIG. 3A-3F). FMAL lifetime decreased for both myosin and actin in the presence of TMR-C0-C2, indicative of binding between C0-C2 and myosin (FIG. 3A) and actin (FIG. 3D) that brings the FRET acceptor (TMR) close enough to FMAL to result in FRET. This effect was reduced when C0-C2 was phosphorylated, consistent with reduced binding (FIGS. 3B and 3E).

Binding curves were generated by calculating the TR-FRET resulting from incubating 1 μM FMAL-labeled myosin or actin with increasing concentrations of TMR-C0-C2 (with the acceptor probes on cys85, cys249, and cys330) (FIG. 4A-4F). PKA phosphorylation of TMR-C0-C2 significantly reduced FRET in both myosin and actin binding assays (FIG. 4A-4F). The EC₅₀, maximum FRET (FRETₘₐₓ), and adjusted R² (adj. R²) were determined by fitting the data to a quadratic model (Table 1). The C0-C2-actin binding curves, both unphosphorylated and phosphorylated (FIG. 4D-4F) fit well to the quadratic model with adj. R²s of 0.99-1.00. Unphosphorylated C0-C2 binding to myosin fit well to the model (adj. R²=0.93-0.96), but the adj. R² of the phosphorylated C0-C2-myosin binding curves were significantly lower (adj. R²=0.60-0.88) (Table 1). The poor fit of the myosin curves with phosphorylated C0-C2 may reflect dramatically decreased or completely abolished binding. FRET between the RLC of myosin and C0-C2 was dependent on RLC's localization to the myosin heavy chain, as 1 μM of unincorporated RLC under the same conditions resulted in <2% FRET (FIG. 6).

TABLE 1
TR-FRET Binding parameters of cC0-C2 binding to myosin
and actin. EC₅₀, FRETₘₐₓ, and Adjusted R² values
for the curves displayed in FIG. 4A-4F are shown.

| | Acceptor | | | | |
|---|---|---|---|---|---|
| | Site | PKA | EC₅₀ (μM) | FRETₘₐₓ (%) | Adj. R² |
| Myosin | C0 | − | 9.0 ± 2.6 | 11.6 ± 1.6 | 0.96 |
| | | + | 125.8 ± 314.6 | 29.5 ± 65.4 | 0.88 |
| | C1 | − | 3.4 ± 1.1 | 12.5 ± 1.4 | 0.93 |
| | | + | 13.2 ± 12.6 | 8.3 ± 4.2 | 0.60 |
| | THB | − | 6.9 ± 2.5 | 12.6 ± 2.0 | 0.93 |
| | | + | 9.7 ± 5.3 | 16.1 ± 15.7 | 0.65 |
| Actin | C0 | − | 1.4 ± 0.2 | 53.2 ± 1.6 | 0.99 |
| | | + | 4.1 ± 0.6 | 47.1 ± 2.6 | 0.99 |
| | C1 | − | 3.0 ± 0.1 | 55.9 ± 0.5 | 1.00 |
| | | + | 5.0 ± 0.5 | 40.8 ± 1.7 | 1.00 |
| | THB | − | 6.3 ± 0.7 | 49.4 ± 2.2 | 0.99 |
| | | + | 17.8 ± 2.8 | 44.3 ± 4.0 | 1.00 |

Binding curve differences due to probe placements: The 3-4-fold differences in EC₅₀ values for probes located in different domains may be due to different binding affinities of the different TMR-C0-C2's for FMAL-actin and FMAL-myosin. As EC₅₀ values are dependent on FRTₘₐₓ values their variability may also be due to variable interactions that influence the saturation level of C0-C2 FRET. This can be influenced by steric interference or cooperative binding and also by different modes of binding that influence the distance between the acceptor probe on C0-C2 and the donor probe on actin or myosin. This is further complicated when there are multiple binding sites on C0-C2 for actin or myosin. These C0-C2 domains can also, in in vitro binding assays, occupy different and multiple binding sites on actin and myosin and bundling of actin filaments indicates that domains within a single C0-C2 molecule can interact independently with multiple actin filaments. There are also multiple binding sites on myosin and actin for C0-C2. Finally, on actin at maximal binding where there is one C0-C2 bound for each actin monomer FRET can take place from TMR-C0-C2 binding to the actin monomer that is labeled or from TMR-C0-C2 on neighboring actin monomers and this can differ depending on the location of the probe. In vivo, this maximum level of binding is of little relevance as C0-C2 is never in competition or close proximity to other C0-C2 molecules. Therefore, the different EC₅₀s may be due to FRET being a measure of both overall binding and where individual binding events take place.

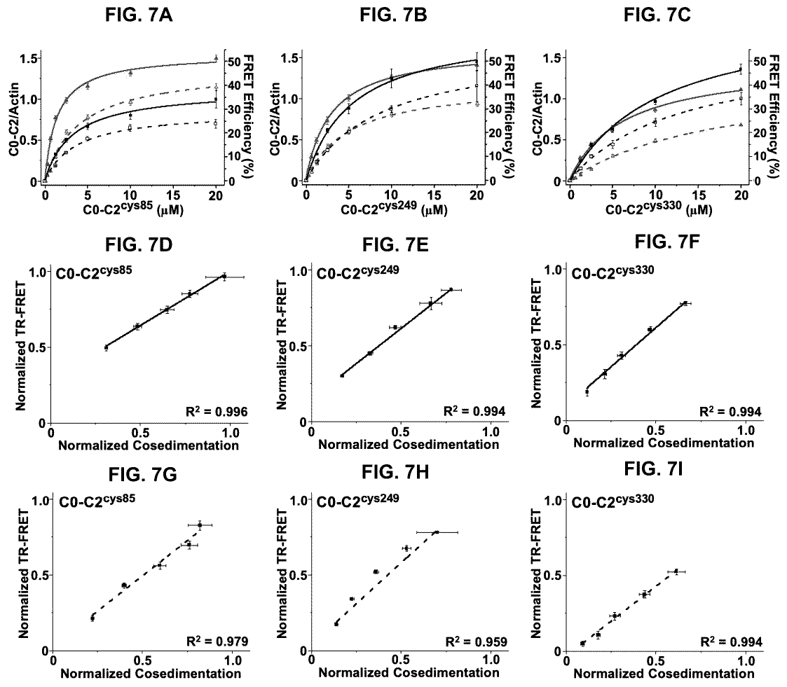

For actin, this has been further explored by doing complimentary cosedimentation assays where binding is due to the combined interactions of the individual C0-C2 domains with actin and individual domain interactions are ignored. Examination of the binding of TMR-C0-C2 with FMAL-actin by cosedimentation suggests that both altered overall affinities and altered modes of binding are factors in the TR-FRET assay. Cosedimentation using unphosphorylated TMR-C0-C2 and FMAL-actin displayed differences in EC₅₀ values that were similar to that observed in the FRET assay, with EC₅₀ values C0-C2^{cys85}<C0-C2^{cys249}<C0_C2^{cys330}. There was good correlation between the two assays (FIG. 7A-7I). This suggests that the FRET differences in binding EC₅₀s are due to overall binding changes due to placing probes in different domains. Comparing the effects of phosphorylation in cosedimentation versus the FRET assays showed significant differences. In cosedimentation assays, phosphorylation of C0-C2^{cys85}, C0-C2^{cys249}, and C0-C2^{cys330}, resulted in a small decrease in overall binding. EC₅₀s were increased 1.1×(C0-C2^{cys85}), 1.6×(C0-C2^{cys249}), and 1.1×(C0-C2^{cys330}). The 1.6× change in overall binding by C0-C2^{cys249} was mirrored by a similar change of 1.7× in the FRET assay. For probes in C0 (C0-C2^{cys85}) and the THB (C0-C2^{cys330}), even though very small changes in overall binding (1.1×) were observed, the FRET changes due to phosphorylation were large (2.9× and 2.8× changes in EC₅₀). This suggests that although binding of unphosphorylated C0-C2 to actin was similar overall (cosedimentation data), the positions of C0 and the THB relative to actin changed such that they were further away from the FMAL on actin when C0-C2 is phosphorylated.

For the analysis of mutant effects, 2.5 μM TMR-C0-C2 were focused on. The FRET effects upon binding to actin are similar for 2.5 μM C0-C2^{cys85} and C0-C2^{cys249} and reduced by 52% upon phosphorylation. TMR-C0-C2^{cys330} effects were about % that observed for the other 2 probe sites and the effect of phosphorylation was a 67% reduction in FRET. Cosedimentation results at 2.5 μM C0-C2 indicate more similar overall binding (43%-61%) when not phosphorylated and (29-37%) when phosphorylated. FRET at TMR-C0-C2^{cys330} (in the THB) displays a large relative difference from the cosedimentation values as was seen when using EC₅₀ values for comparison. The effects of the M-domain on muscle function are mediated by both phosphorylated region and the THB and the results of using either the values at 2.5 μM or EC₅₀s for comparison of FRET and cosedimentation demonstrates that the interactions of the THB with actin is more sensitive to phosphorylation than overall binding of C0-C2. The EC₅₀ comparisons, but not those at 2.5 μM, suggest that the same is true for the binding of C0 that is separated from the phosphorylated M-Domain by the PAL, and C1. Altered positioning of C0 is consistent with experiments that found changes in C0-C1 spatial relationships upon phosphorylation of C0-C2. Therefore, the FRET assay displayed sensitivity to the mode of binding that is missing in cosedimentation assays.

Comparing phosphorylation effects between probe locations either using EC₅₀ values (Table 4) or binding levels at 2.5 μM indicates that C0 is the most sensitive and the THB the least. This indicates a large, combined effect of binding and spatial relationship of C0 to RLC upon phosphorylation and less of change in the spatial relationship of the THB to RLC. This is consistent with binding studies that found C0 bound to RLC.

High-throughput screening (HTS) potential of TR-FRET C0-C2-myosin and C0-C2-actin binding assays: To quantitatively illustrate the suitability of the myosin and actin TR-FRET binding assays in HTS for compounds that reduce C0-C2 binding, the average and standard deviation values for the lifetimes of FMAL-myosin and FMAL-actin measured in the absence and presence of 5 μM TMR-C0-C2^{cys249} (for FRET values at this C0-C2 concentration, see arrows in FIG. 4A-4F) were used to calculate a Z′-factor (see Eq. 3). Results from 4 independent preparations gave Z′-factor ranges of 0.76-0.90 for myosin and 0.94-0.95 for actin, classifying them as “excellent” for use in HTS for compounds that reduce cMyBP-C interactions with myosin or actin (Table 2). PP3T.

TABLE 2
Z′-factor test for TMR-C0-C2 effects on FMAL-myosin and FMAL-actin lifetimes:
Lifetimes of 1 μM FMAL-myosin and 1 μM FMAL-actin in the absence (−) and
presence (+) of 5 μM TMR-C0-C2^{cys249}. Data is shown for 3-5 wells in each
condition from 4 independent protein preps. Z′-factors (Z′) for each prep
comparing lifetimes in the absence and presence of C0-C2^{cys249} are shown.

| | Prep | C0-C2 | Lifttimes (ns) | | | | | AVG | STD | Z′ |
|---|---|---|---|---|---|---|---|---|---|---|
| Myosin | 1 | − | 3.66 | 3.66 | 3.67 | 3.67 | 3.67 | 3.67 | 0.005 | 0.76 |
| | | + | 3.51 | 3.51 | 3.51 | 3.50 | 3.52 | 3.51 | 0.007 | |
| | 2 | − | 3.50 | 3.51 | 3.51 | 3.50 | 3.52 | 3.51 | 0.008 | 0.84 |
| | | + | 3.25 | 3.25 | 3.25 | 3.26 | 3.25 | 3.25 | 0.006 | |
| | 3 | − | 3.43 | 3.42 | 3.44 | 3.43 | 3.43 | 3.43 | 0.005 | 0.85 |
| | | + | 3.24 | 3.25 | 3.24 | 3.24 | 3.24 | 3.24 | 0.004 | |
| | 4 | − | 3.79 | 3.78 | 3.80 | 3.80 | 3.80 | 3.79 | 0.005 | 0.90 |
| | | + | 3.52 | 3.53 | 3.52 | 3.52 | 3.52 | 3.52 | 0.004 | |
| Actin | 1 | — | 4.08 | 4.07 | 4.09 | − | − | 4.08 | 0.007 | 0.94 |
| | | + | 2.85 | 2.82 | 2.84 | − | − | 2.84 | 0.018 | |
| | 2 | − | 4.20 | 4.21 | 4.18 | 4.21 | 4.21 | 4.20 | 0.013 | 0.95 |
| | | + | 2.64 | 2.65 | 2.63 | 2.62 | 2.64 | 2.64 | 0.011 | |
| | 3 | − | 3.81 | 3.80 | 3.80 | 3.80 | 3.79 | 3.80 | 0.007 | 0.95 |
| | | + | 2.44 | 2.40 | 2.42 | 2.43 | − | 2.42 | 0.017 | |
| | 4 | − | 3.81 | 3.80 | 3.80 | 3.80 | 3.79 | 3.80 | 0.007 | 0.95 |
| | | + | 2.44 | 2.40 | 2.42 | 2.43 | − | 2.42 | 0.021 | |

Myosin and Actin Binding curve differences: C0-C2 appears to bind closer to actin cys374 than to RLC^{cys105} in myosin. FRETₘₐₓ values describe the maximum level of FRET efficiency for each binding curve and is dependent on the distance between the FRET donor and acceptor probe and the R₀ of the probe pair. For the FMAL-TMR probe pair (R₀=4.9-5.6 nm), 100% FRET is expected if the probes are separated by 0-2.2 nm, and beyond 11 nm no FRET is expected. For unphosphorylated C0-C2 levels, myosin FRETₘₐₓ values were between ˜11-12% FRET, whereas actin TR-FRET showed values between ˜49-56% FRET. The higher levels of FRET from C0-C2-actin binding can be interpreted as closer proximity of the donor probe on the actin filament (versus myosin) to the acceptor probes on C0-C2. It is also possible for C0-C2 bound to neighboring actin monomers to display FRET with one labeled FMAL-actin and this may further explain the high FRET levels seen for actin binding.

R² values, a measure of the goodness of fit to the quadratic model, are quite good for actin TR-FRET binding data for both unphosphorylated and phosphorylated C0-C2 (R²=0.99-1.00). In the myosin-C0-C2 TR-FRET binding assay, the unphosphorylated C0-C2 for each probe site indicated that the model was a good fit (R²=0.93-0.96). However, the curves generated for phosphorylated C0-C2 in myosin TR-FRET showed poor fit to the model (R²=0.60-0.88). This may be due to the inability to reach FRET saturation in the phosphorylated C0-C2 curves at these concentrations. This is consistent with cosedimentation experiments that found a binding affinity of 2.8 μM for C0-C2 binding to myosin, but reliable fittings were unobtainable for C0-C2 that had phosphomimetic mutations at the PKA phosphorylation sites. MST binding studies with C1-C2 and myosin ΔS2 and C1-C2 cosedimentation studies with native thin filaments also found that phosphorylation reduced binding to thin filaments and abolished its binding to ΔS2. The relative effects of phosphorylation on MyBP-C binding to actin and myosin may be critical given that the very high local concentration of all 3 proteins in the sarcomere suggests that C0-C2 is always bound to either actin or myosin. In a competition model, where C0-C2 is always bound to either the thin or thick filament, phosphorylation might reduce C0-C2 affinity to actin but, due to eliminating (or drastically reducing) its binding to myosin, the equilibrium between thick and thin filaments may be shifted towards more actin binding of the phosphorylated N-terminal domains. In this case, increased binding to actin does not imply increased effects on the thin filament as phosphorylated MyBP-C binding to the thin filaments has very reduced effects on myofilament functions when compared to unphosphorylated MyBP-C.

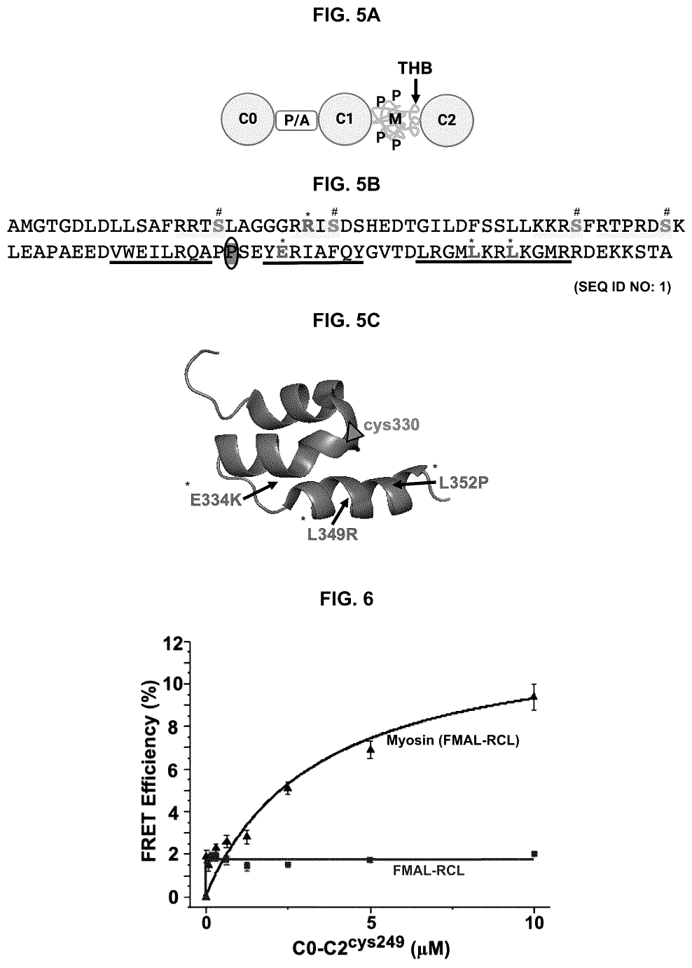

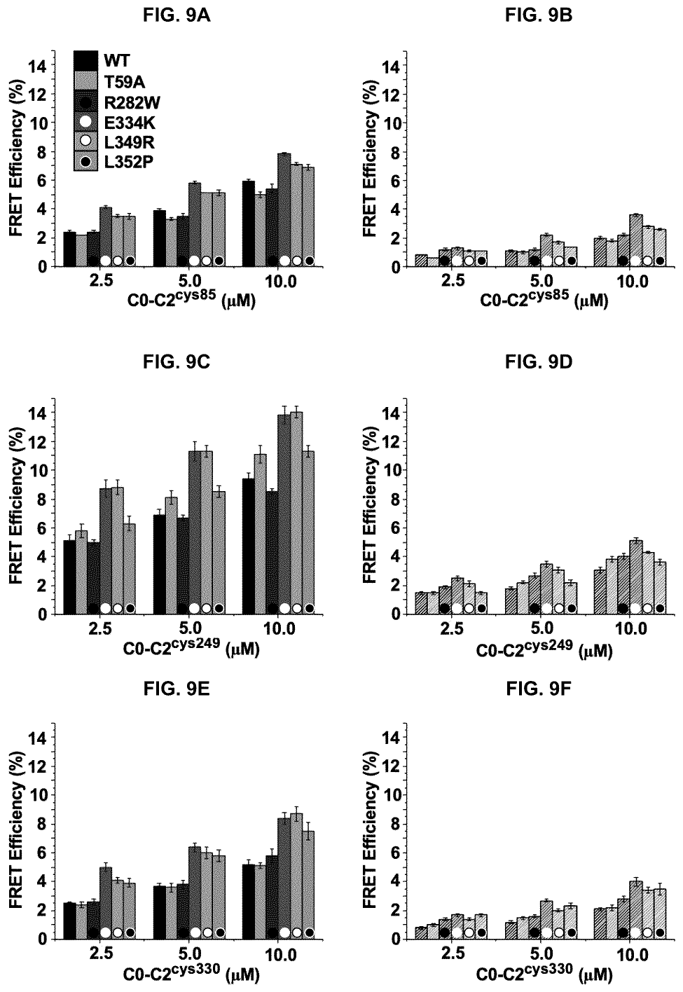

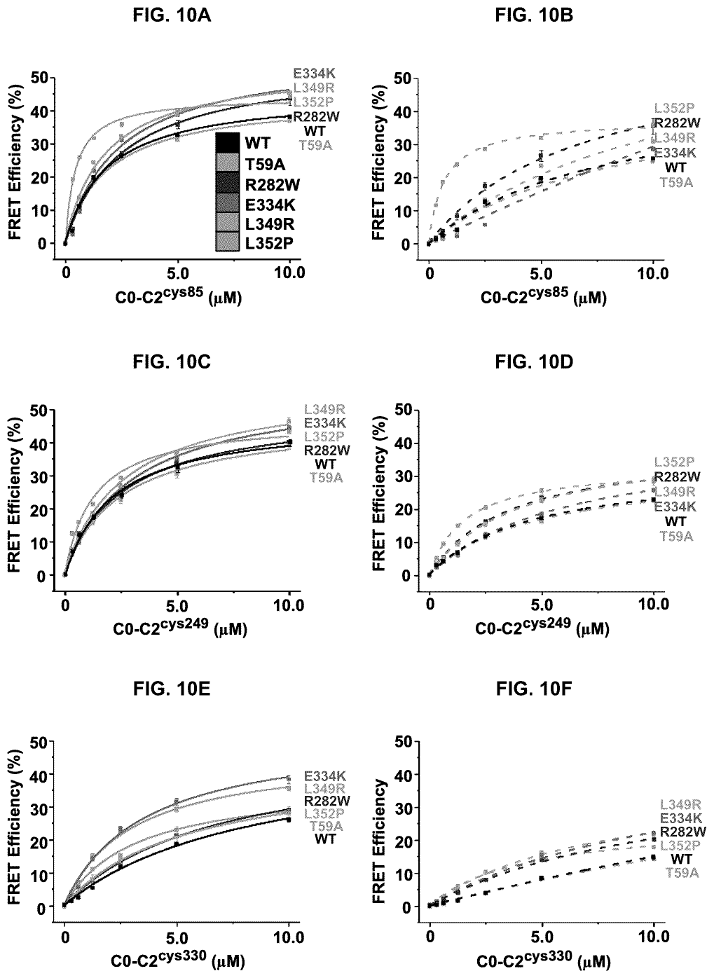

C0-C2 HCM mutation effects on myosin and actin TR-FRET: The TR-FRET assays were used to assess the effects of 5 N-terminal MyBP-C mutations, one in C0, one in a PKA phosphorylation target sequence and 3 in the THB located in the M-domain (FIG. 5A-5C). These five mutations were introduced into C0-C2 proteins that contained each of the 3 acceptor probe sites, for a total of 15 mutant proteins. These 15 TMR-labeled mutants and 3 wild-type C0-C2 proteins were combined with FMAL-labeled myosin and actin, and the resulting TR-FRET was measured. Table 3 shows the relative TR-FRET resulting from 2.5 μM C0-C2 for each mutation, unphosphorylated and phosphorylated, interacting with myosin or actin compared to that of wild type C0-C2. The concentration, 2.5 μM C0-C2, shown in Table 3, shows clear submaximal binding (FIG. 4A-4F, arrowheads). TR-FRET at this concentration has the capacity to detect increases and decreases in binding due to mutations. TR-FRET values observed with labeled myosin at 2.5, 5, and 10 μM C0-C2 can be found in FIG. 9A-9F (myosin) and complete binding curves for actin are found in FIG. 10A-10F (actin).

T59A, in C0, at 2.5 μM C0-C2, displayed FRET values that were within 20% of that found for wild type C0-C2 in 5 of the 6 conditions tested (3 probe acceptor sites, minus and plus phosphorylation) for both actin and myosin. On myosin, the probe in the THB (cys330) did show a 24% increase in FRET with myosin when T59A was phosphorylated and a 21% increase in FRET with actin in the unphosphorylated state (Table 3).

R282W, in an M-domain PKA target sequence, altered phosphorylation and actin interactions. New FRET assays described herein found no effects on the binding of unphosphorylated R282W to either myosin or actin. FRET was within 10% of wild type for all 3 FRET acceptor probes. The phosphorylated TMR-C0-C2s with R282W all displayed increased FRET with both myosin (24%-64% increases) and actin (37%-105% increases). The largest effects on FRET for phosphorylated R282W with both myosin (64%) and actin (105%) were observed when the acceptor probe was located in the THB at cys330 (Table 3).

E334K, in the THB, increased myosin FRET in all 6 conditions by 66%-100%. With actin, probes on C0 and C1 showed little change (within 18% of wild type) or a significant decrease to 45% of wild type FRET for the C0 probe when phosphorylated. When the probe is in the THB, E334K, unphosphorylated and phosphorylated, promotes a doubling of the FRET with actin (Table 3).

L349R consistently increases myosin FRET by 43%-72% in all conditions tested. Actin FRET with L349R gave mixed results with probes on C0 and C1. Minimal change in phosphorylated C0 and unphosphorylated C1 (4% and 10%, respectively). Moderate changes were observed in unphosphorylated C0 and phosphorylated C1 (22% and 31%, respectively). The probe on the THB responded strongly to L349R with an increase of 89% (unphosphorylated) and 166% (phosphorylated) (Table 3).

L352P increases both myosin and actin FRET. With both myosin and actin, the effects were largest for the probe in the THB, followed by the probe in C0 and then the one in C1. On actin, large effects (74%-160%) were observed for all probe sites when L352P was phosphorylated. For myosin, the phosphorylated form showed a large effect (101% increase) on the probe in the THB, a moderate effect (36% increase) on the probe in C0, and no effect on the probe in C1 (Table 3).

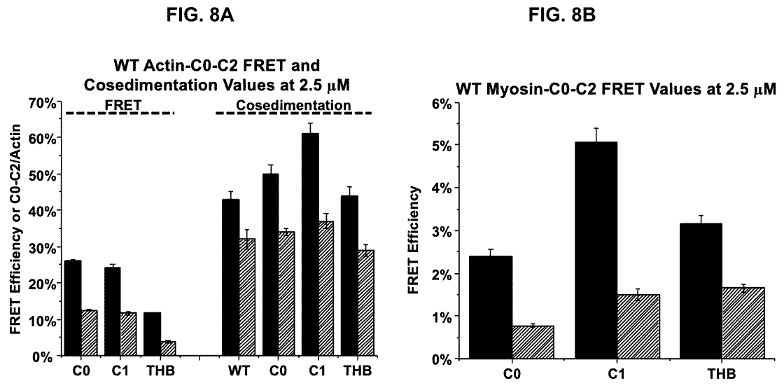

TABLE 3
Effects of C0-C2 mutations on TR-FRET measured binding to myosin
and actin: Mutations T59A, R282W, E334K, L349R, and L352P in
C0-C2 with TMR FRET-acceptor in C0 (TMR-C0-C2cys85), C1 (TMR-
C0-C2cys249), or THB-domain (TMR-C0-C2cys330) were tested with
1 μM of FMAL-myosin and FMAL-actin. The FRET value of each
HCM mutation at 2.5 μM relative to that observed for wild
type (WT) C0-C2 is shown for each probe site. For WT FRET values
at 2.5 μM C0-C2 see FIG. 8A and 8B. * indicates <50% of
WT, plain text indicates a 20% decrease or increase in FRET
relative to wild type, bold text indicates a 20% to 50% increase,
bold italicized text indicates a 50% to 100% increase, and bold
underlined text indicates >100% increase (N = 2-3, n = 8-15).

| C0-C2 | | | | | | | |
|---|---|---|---|---|---|---|---|
| Acceptor | | Myosin | | | Actin | | |
| Site | PKA | C0 | C1 | THB | C0 | C1 | THB |
| T59A | − | 0.91 | 1.15 | 0.96 | 0.97 | 0.92 | 1.21 |
| | + | 0.80 | 1.04 | 1.24 | 0.89 | 0.96 | 0.98 |
| R282W | − | 1.01 | 0.98 | 1.02 | 1.03 | 0.97 | 1.10 |
| | + | [symbol] | 1.24 | [symbol] | 1.37 | 1.37 | 2.05 |
| E334K | − | [symbol] | [symbol] | [symbol] | 1.18 | 1.08 | [symbol] |
| | + | [symbol] | [symbol] | [symbol] | 0.45* | 1.05 | 2.03 |
| L349R | − | 1.45 | [symbol] | [symbol] | 1.22 | 1.10 | [symbol] |
| | + | 1.45 | 1.43 | [symbol] | 1.04 | 1.31 | 2.66 |
| L352P | − | 1.46 | 1.25 | [symbol] | 1.36 | 1.21 | 1.31 |
| | + | 1.36 | 0.99 | 2.01 | 2.27 | [symbol] | 2.60 |

| | | | | | |
|---|---|---|---|---|---|
| Relative to WT FRET | *<0.5 | 0.8-1.2 | 1.2-1.5 | [symbol] | >2.0 |
| at 2.5 μM | | | | | |

Effects of 5 C0-C2 mutations on myosin and actin binding: Five HCM-linked or function altering mutations were introduced into the 3 C0-C2 FRET acceptor proteins and their effects on myosin- and actin-based FRET were determined. FRET results at 2.5 μM of C0-C2 binding to myosin and actin, in the absence or presence of PKA phosphorylation, compared with wild type C0-C2 showed clear effects of the mutations that further illustrate the usefulness and potential of these assays for classifying mutations based on binding abilities. 2.5 μM of C0-C2 is below maximal binding and can readily detect increases and decreases of FRET for all three constructs with probes located in the C0, C1, and THB of the M-domain (arrowheads in FIG. 4A-4F, FIGS. 9A-9F, and FIG. 10A-10F). The mutations tested were in the C0-domain (T59A) and the M-domain (R282W, in a PKA phosphorylation recognition site, and E334K, L349R, L352P in the THB).

Myosin filament preparations: Cardiac myosin from porcine ventricles (Pel Freeze) was prepared and further purified with ion-exchange chromatography (HiPrep DEAE FF 16/10 column) to remove contamination of thin filament proteins and MyBP-C. The column was equilibrated with, and myosin applied in, 40 mM NaPyrophosphate pH 7.5. Myosin was eluted with a gradient of 0-500 mM KCl, 20 mM NaPyrophosphate pH 7.5, over 100 mL at 2 mL/min, collecting 5 mL fractions (AKTA Prime Plus, GE). SDS-page was used to pool the fractions with <2% of actin contamination. The final myosin was dialyzed into 600 mM KCl, 25 mM KPᵢ, 2 mM DTT, pH 7.0 buffer. For storage at −80° C., sucrose was added to 150 mM (final concentration), and flash frozen in a dropwise manner into liquid nitrogen.

RLC preparation and labeling: Human RLC (from the myosin light chain 2 gene, MYL2) coding sequence with E. coli optimized codons was inserted into the pET45b expression vector (GenScript). Mutagenesis of cys105 to cysteine was performed using a Q5 Site-Directed Mutagenesis Kit (New England Bio Labs). The sequence was confirmed by DNA sequencing (Eton Biosciences, San Diego, CA). For protein production, the RLC expression plasmid was transformed into E. coli BL21DE3-competent cells (New England Bio Labs, Ipswich, MA), and grown at 37° C., in Overnight Express Autoinduction medium (Novagen, Madison, Wisconsin) supplemented with 100 μg/ml ampicillin. RLC was purified by inclusion body isolation followed by ion exchange chromatography. Bacteria were harvested by centrifugation for 5 min., 4° C., 8,000×g, resuspended in phosphate buffered saline (154 mM NaCl, 0.8 mM KH2PO4, 5.6 mM Na₂HPO₄ pH 7.4) (5-10 ml/g cell paste) containing 0.1 mM PMSF, collected again by centrifugation for 20 min., 4° C., 3,300×g, and frozen at −80° C., until use. The frozen bacterial cell pellet was resuspended in lysis buffer (25 mM Tris-Cl, 5 mM EDTA, 1 mM PMSF, 50 mM glucose, pH 8.0) and disrupted using an Emulsiflex homogenizer (Avestin, Ottowa, Ontario). MgCl₂ was added (final concentration 10 mM0), followed by the addition of DNAse 1 (final concentration 5 mg/ml) and incubation at 4° C. for 1 hour with rocking. Triton X-100 was added (from a 10% stock) to a final concentration of (0.1%). To collect insoluble proteins, including RLC in inclusion bodies, the lysed bacterial were centrifuged 15 min, 4° C., 20,000×g in a Beckman JA-17 rotor. The inclusion bodies were washed 2× by resuspension in lysis buffer plus Triton X-100 (0.1%) followed by centrifugation and finally washed with lysis buffer without Triton X-100. The resulting pellet was frozen at −20° C. until further purification. RLC inclusion bodies collected from 1 L of bacterial culture were resuspended in 20 ml resuspension buffer (7 M Urea, 30 mM Tris-HCl, 50 mM NaCl, pH 7.5, and 1 mM DTT (final concentration added just prior to use)). This was stirred at room temperature (23° C.) for 30 minutes or until the pellet was completely dissolved. Any remaining insoluble debris was removed by centrifugation 30 min, 4° C., 30,000×g in a Beckman JA-17 rotor. The supernatant containing denatured RLC was applied to an ion exchange column (Hitrap_Q_XL_5 mL). The column was first equilibrated with 4 M Urea, 30 mM Tris-HCl, 50 mM NaCl, 1 mM DTT, and pH 7.5. RLC was eluted in a gradient of 50-250 mM NaCl (and 4 M Urea, 30 mM Tris-HCl, 1 mM DTT, pH 7.5) over 50 mL at 1 mL/min, collecting 1 mL fractions (AKTA Prime Plus, GE). Fractions were analyzed by SDS-PAGE, and those containing pure RLC were pooled and dialyzed into 0.4 mM KCl. The concentration was adjusted to 250 mM, flash frozen in liquid nitrogen, and stored at −80° C. until use.

Labeling of 50 μM RLC with fluorescein-5-maleimide (FMAL, Invitrogen #F150) was done in TUKE buffer (50 mM Tris, 6 M Urea, 80 mM KCl, 1 mM EDTA, pH 7.5). Since frozen RLC is 250 mM in 0.4 mM KCl, it was diluted 5× with 1.25× TUKE buffer lacking KCl (62.5 mM Tris, 7.5 M Urea, 1.25 mM EDTA, pH 7.5). TCEP was added (200 μM final concentration) and incubated with the RLC for 30 min at 23° C. followed by the addition of FMAL (from a 20 mM stock in DMF stored at −80° C.) to a final concentration of 275 μM. Labeling was done for 2 h at 23° C. and terminated by the addition of DTT (to a final concentration of 1.5 μM). Unincorporated dye was removed by extensive dialysis into the exchange buffer (50 mM NaCl, 5 mM EDTA, 2 mM EGTA, 10 mM NaPO4, pH 7.5). Following dialysis, labeled RLC was centrifuged for 30 min at 100,000 RPM (350,000×g) in a Beckman TLA-120.2 rotor to remove any precipitated dye and insoluble protein. The degree of labeling, typically 60-80%, was determined by using FMAL's extinction coefficient (E494=68,000 cm⁻¹M⁻¹), and the concentration of RLC determined by using a BCA assay with BSA as a standard.

RLC exchange onto myosin: RLC exchange onto myosin was adjusted for the use of full-length myosin. Labeled RLC and purified myosin, in exchange buffer containing 1 mM DTT, were mixed to final concentrations of 25 μM of 10% labeled RLC (2.5 μM FMAL labeled RLC, 22.5 μM unlabeled RLC) and 5 μM of myosin. ATP was added to an FC of 2 mM. After 50 min incubation at 42° C., MgCl₂ (from a 500 mM stock) was slowly added to 15 mM to stop the exchange. To remove excess RLC, the mixture was centrifuged at 200,000×g in a Beckman TLA-120.2 rotor, pelleting the myosin filaments. The supernatant containing excess RLC was removed, and the pellet was washed with an exchange buffer containing 15 mM MgCl₂. The pellet was centrifuged at 200,000×g for an additional 5 min in the wash solution. The supernatant was removed, and the final pellet was resuspended in 600 mM NaCl, 0.2 mM EDTA, 25 mM Tris, pH 7.0, clarified by centrifugation at 4° C., 15,000 rpm (21,000×g) for 10 min in an Eppendorf 5424R benchtop microfuge and then dialyzed into 75-20-3 buffer (75 mM KCl, 20 mM Tris, 3 mM MgCl₂, 1 mM DTT, pH 7.0) for TR-FRET experiments.

Actin filament preparations and labeling: Actin was prepared from rabbit skeletal muscle by extracting acetone powder in cold water. Actin was labeled as F-actin. 50 μM G-actin (in 10 mM Tris, 0.2 mM CaCl₂, 0.2 mM ATP, pH 7.5) was brought to 20 mM Tris (pH 7.5) and then polymerized by the addition of MgCl₂ to a final concentration of 2 mM and KCl to a final concentration of 100 mM. Polymerization was allowed to proceed for 1 hour at 23° C. FMAL was added to a final concentration of 0.5 mM (from a 20 mM stock in DMF stored at −80° C.). Labeling was for 1 h at 23° C. and stopped by the addition of DTT to 2.5 mM. Labeled F-actin was collected by centrifugation (30 min, 100,000 rpm (350,000×g) in a Beckman TLA-120.2 rotor at 4° C.). The F-actin pellet was rinsed 3× with actin labeling G-buffer (5 mM Tris, 0.2 mM CaCl₂, 0.5 mM ATP, pH 7.5) containing 3 mM MgCl₂ then 1× with actin labeling G-buffer and then resuspended in actin labeling G-buffer. Labeled G-actin was clarified to remove any remaining F-actin or precipitated FMAL by centrifugation (10 min, 90,000 rpm in a Beckman TLA-120.2 rotor at 4° C.). The extent of labeling (typically around 70%) was determined by UV-vis spectrophotometry. Labeled G-actin was mixed with unlabeled G-actin to achieve a 10% labeled mixture. This was polymerized by the addition of MgCl₂ to a final concentration of 2 mM and KCl to a final concentration of 100 mM and dialyzed against MOPS actin-binding buffer (M-ABB; 100 mM KCl, 10 mM MOPS, pH 6.8, 2 mM MgCl₂, 0.2 mM CaCl₂, 0.2 mM ATP, 1 mM DTT, 1 mM sodium azide). Any bundled actin was removed by centrifugation at 4° C., 15,000 rpm (21,000×g) for 10 min in an Eppendorf 5424R benchtop microfuge. Actin concentration and % FMAL labeling were again determined by UV-vis spectrophotometry. The labeled F-actin was stabilized with the addition of phalloidin (to the same concentration as actin).

Recombinant human cMyBP-C and labeling: pET45b vectors encoding E. coli optimized codons for the C0-C2 portion of human cMyBP-C with N-terminal 6× His tag and TEV protease cleavage site were obtained from GenScript. C0-C2 mutants were engineered using a Q5 Site-Directed Mutagenesis Kit (New England Bio Labs). Substitution mutations were performed to generate C0-C2 constructs containing a single cysteine located in the C0-domain at position 85, C1-domain at position 249, or the THB in the M-domain at position 330 (termed C0-C2^{cys85}, C0-C2^{cys249}, and C0-C2^{cys330}). Five endogenous cysteines were removed to generate C0-C2^{cys85} and C0-C2^{cys330} by introducing the following mutations: C239L, C249S, C426T, C436V, and C443S (32). To insert the cysteine of interest for probe labeling, mutation S85C was inserted in C0-C2^{cys85} and mutation P330C was inserted in C0-C2^{cys330}. Since C0-C2^{cys249} contains an endogenous cysteine at position 249, the remaining four endogenous cysteines were similarly removed. The following N-terminal cMyBP-C HCM mutations were introduced to each C0-C2^{cys85}, C0-C2^{cys249}, and C0-C2^{cys330} construct: T59A, R282W, E334K, L349R, and L352P. The results were C0-C2 constructs containing a single cysteine in the C0, C1, or M-domain with an cMyBP-C HCM mutation inserted: C0-C2^{cys85, T59A}, C0-C2^{cys85, R282W}, C0-C2^{cys85, E334K}, C0-C2^{cys85, L349R}, C0-C2^{cys85, L352}, C0-C2^{cys249, T59A}, C0-C2^{cys249, R282W}, C0-C2^{cys249, E334K}, C0-C2^{cys249, L349R}, C0-C2^{cys249, L352P}, C0-C2^{cys330, T59A}, C0-C2^{cys330, R282W}, C0-C2^{cys330, E334K}, C0-C2^{cys330, L349R}, C0-C2^{cys330, L352P}. All sequences were confirmed by DNA sequencing (Eton Biosciences). Protein production in E. coli BL21DE3-competent cells (New England Bio Labs) and purification of C0-C2 protein using His60 Ni Superflow resin were done as described (40). C0-C2 (with His-tag removed by TEV protease digestion) was further purified using size-exclusion chromatography to achieve >90% intact C0-C2 as described (26) and then concentrated, dialyzed to 50/50 buffer (50 mM NaCl and 50 mM Tris, pH 6.7), and stored at 4° C. For long term storage at −20° C., glycerol, containing 50 mM NaCl and 50 mM Tris, pH 6.7, was added to 50%.

C0-C2 was labeled with tetramethylrhodamine-5-maleimide (TMR, AnaSpec. Inc, #AS-s81446) in 50/50 buffer. C0-C2 (50 μM) was first treated with TCEP (200 μM) for 30 min at 23° C. while rocking. TMR (stored as 20 mM stock in DMF at −80° C.) was added to final concentrations of 75 μM (C0-C2^{cys85} constructs), 125 μM C0-C2^{cys249} constructs) 140 μM C0-C2^{cys330} constructs). Labeling was done for 1 h at 23° C. and terminated by the addition of 5× molar excess of DTT. Unincorporated dye was removed by extensive dialysis against 75-20-3 buffer (for myosin binding) or M-ABB buffer (for actin binding). Following the dialysis, labeled C0-C2 was centrifuged for 30 min at 100,000 RPM (350,000×g) in a Beckman TLA-120.2 rotor to remove any precipitated dye and insoluble protein. The degree of labeling ranged from 50-90% dye/C0-C2 as measured by UV-vis absorbance. To attain uniform 50-60% dye/C0-C2 for TR-FRET experiments, unlabeled corresponding C0-C2 construct was added.

Protein and Dye Concentration: The Bradford protein concentration assay (BCA) and SDS-Page Gel using a BSA protein standard was used throughout this study to determine protein concentrations. The extinction coefficient for FMAL is 68,000 at 494 nm and the extinction coefficient for TMR is 91,000 at 542 nm, provided by manufacturer's specifications.

In vitro phosphorylation of cMyBP-C: C0-C2 was treated with 7.5 ng PKA/μg C0-C2 at 30° C. for 30 min. This is 3× the level (2.5 ng PKA/μg C0-C2) needed to achieve maximal phosphorylation as determined by in-gel staining of proteins with Pro-Q Diamond.

Steady-state basal and actin-activated ATPase activity of myosin: NADH-coupled assay was used to measure the steady-state ATPase activity of myosin. The assay was performed at 23° C. in buffer containing 10 mM KCl, 4 mM MgCl₂, 20 mM Tris-HCl, and at pH 7.5 Each reaction contained 1 μM of myosin±4 μM of actin and 2 mM phosphoenolpyruvate, 0.3 mM NADH, 38 U/ml pyruvate kinase, 50 U/mL lactate dehydrogenase, and 2 mM MgATP. Changes in NADH absorption, at 340 nm, was measured over 15 min using a Beckman DU730 UV-Vis spectrophotometer. The rates, ADP myosin⁻¹s⁻¹, (commonly expressed as s⁻¹) were derived from this data.

Time-Resolved FRET (TR-FRET) data acquisition and analysis: Fluorescence lifetime measurements were acquired using a high-precision fluorescence lifetime plate reader (FLTPR; Fluorescence Innovations, Inc), provided by Photonic Pharma LLC. For TR-FRET experiments, FMAL was excited with a 473-nm microchip laser (Bright Solutions) and emission was filtered with 488-nm long-pass and 517/20-nm band-pass filters (Semrock). The photomultiplier tube (PMT) voltage was adjusted so that the peak signals of the instrument response function (IRF) and the biosensor were similar (˜100 mV). The observed waveforms were convolved with the IRF to determine the lifetime (T) (Eq. 1) by fitting to a single-exponential decay. The decay of the excited state of the fluorescence FMAL dye attached to myosin or actin to the ground state is:

$$F ( t ) = I 0 exp exp ( - \frac{t}{τ} ) \qquad ( Eq . 1 )$$

where I0 is the fluorescence intensity upon peak excitation (t=0), and τ is the fluorescence lifetime (t=τ when I decays to 1/e or ˜37% of I0). The efficiency of energy transfer E (FRET efficiency) was calculated as:

$$E = 1 - ( \frac{τ D A}{τ D D} ) \qquad ( Eq . 2 )$$

Where T_{DA} is the lifetime of FMAL-Myosin or FMAL-Actin in the presence of TMR-C0-C2 and T_{D} is the average lifetime of FMAL-Myosin or FMAL-Actin in the absence of C0-C2.

Determination of FRETₘₐₓ and EC₅₀ values: The maximum FRET (FRETₘₐₓ) and EC₅₀ values for C0-C2 binding to myosin and actin were determined by fitting the data to a quadratic model (Michaelis-Menten function) using Origin Pro 2019 computer software package through a nonlinear least-squares minimization (Levenberg-Marquardt algorithm) as previously described in Bunch et al. (40, 58). These EC₅₀ and FRETₘₐₓ values are used as comparative indicators of binding characteristics for C0-C2 binding to myosin and actin under different conditions (±phosphorylation or in the presence of mutations). The adjusted R² was used to test the goodness-of-fit to the model.

Determination of Z′-Factor for C0-C2^{cys249}: For suitability in high-throughput screening (HTS), the lifetime of FMAL attached to myosin or actin was determined in multiple wells in the absence or presence of 5 μM TMR-labeled C0-C2^{cys249}. The lifetimes in the absence (T_{A}) and presence of TMR-C0-C2^{cys249} (τ_{B}) were compared and indexed by the Z′-factor (Eq. 3):

$${Z}^{′} = 1 - \frac{3 ( σ A + σ B )}{\vert μ A - μ B \vert} \qquad ( Eq . 3 )$$

where σ_{A} and σ_{B} are the standard deviations (S.D) of the T_{A} and T_{B} lifetimes, respectively, μ_{A} and μ_{B} are the means of the T_{A} and T_{B} lifetimes, respectively. Z′-factor of less than 0 is indicative of “useless” conditions, 0 to 0.5 is “good” and 0.5 to 1.0 is “excellent” assay quality.

Statistics: Average data are provided as mean±standard error (SE). Each experiment was done with >2 separate protein preparations. Statistical significance (p<0.05) is evaluated by use of Student's t-test. Biological repeats (N, independent protein preparations) and technical repeats (n, independent reactions) are as indicated in each figure and table.

TABLE 4
Cosedimentation Binding parameters of cC0-C2 binding to
actin. EC₅₀, Bₘₐₓ, and adjusted R² values for the cosedimentation
curves displayed in FIG. 7A-7I are shown.

| | Acceptor | | | Bₘₐₓ | |
|---|---|---|---|---|---|
| | Site | PKA | EC₅₀ (μM) | (C0-C2/Actin) | Adj. R² |
| Actin | C0 | − | 3.27 ± 0.40 | 1.12 ± 0.04 | 0.99 |
| | | + | 3.66 ± 0.50 | 0.85 ± 0.04 | 0.99 |
| | C1 | − | 5.40 ± 0.42 | 1.86 ± 0.06 | 1.00 |
| | | + | 8.72 ± 0.43 | 1.64 ± 0.04 | 1.00 |
| | THB | − | 10.58 ± 1.34 | 2.05 ± 0.13 | 1.00 |
| | | + | 12.80 ± 1.14 | 1.63 ± 0.08 | 1.00 |

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of using time-resolved fluorescence energy transfer (TR-FRET) and a fluorescent protein biosensor to quantitate protein binding in solution, the method comprising:
a) labeling a contractile protein with a first fluorescent probe and labeling a myosin binding protein-C (MyBP-C) with a second fluorescent probe to generate a fluorescent protein biosensor suitable for TR-FRET;
b) measuring FRET efficiency when structural changes in the fluorescent protein biosensor occur, wherein FRET efficiency is a proportion of donor molecules that have transferred excitation state energy to acceptor molecules; and
c) quantitating protein binding using the measured FRET efficiency.

2. The method of claim 1, wherein the contractile protein and the MyBP-C operably connect or bind in a physiological solution.

3. The method of claim 1, wherein the contractile protein comprises an actin protein, or a myosin protein, wherein the myosin protein comprises a full-length myosin protein or a fragment thereof, wherein the fragment of the myosin protein comprises a heavy meromyosin (HMM), a myosin subfragment 1 (S1), a myosin regulatory light chain (RLC) or a myosin subfragment 2 (S2); wherein the actin protein comprises an F-actin protein or a fragment thereof.

4. The method of claim 1, wherein the MyBP-C comprises a full-length MyBP-C or a fragment thereof, wherein the fragment of the MyBP-C comprises a C0-C2 fragment, a C0-C2 fragment, or a C0-C7 fragment.

5. The method of claim 1, wherein the first fluorescent probe comprises a fluorescent donor probe and the second fluorescent probe comprises a fluorescent acceptor probe; wherein the FRET efficiency is the proportion of donor molecules from the fluorescent donor probe that have transferred excitation state energy to acceptor molecules on the fluorescent acceptor probe.

6. The method of claim 1, wherein the contractile protein comprises one or more point mutations.

7. The method of claim 1, wherein the second fluorescent probe is attached to a C1 domain, a C3 domain, a C4 domain, a C5 domain, or a combination thereof of the MyBP-C.

8. The method of claim 7, wherein the second fluorescent probe is attached to a C249 residue or an H225 residue within the C1 domain, a C528 residue within the C3 domain, a C623 residue within the C4 domain, a C719 residue within the C5 domain, or a combination thereof.

9. The method of claim 1, wherein the MyBP-C further comprises one or more genetic modifications.

10. The method of claim 9, wherein the one or more genetic modifications are mutations associated with a disease, wherein the disease is heart failure or hypertrophic cardiomyopathy (HCM).

11. The method of claim 9, wherein the one or more genetic modifications comprise E334K, L349R, and L352P.

12. The method of claim 1, wherein the first and second fluorescent probes are selected from a group consisting of IAEDANS, IAANS, CPM, IANBD, 5-IAF, TMP, ATTO FMAL, Alexa Fluor 488, Alexa Fluor 532, and Alexa Fluor 568.

13. The method of claim 1, wherein the method is ATP-free.

14. An in vitro method for identifying drug candidates for treating hypertrophic cardiomyopathy and/or heart failure using time-resolved fluorescence energy transfer (TR-FRET) and a fluorescent protein biosensor to quantitate structural changes of the fluorescent protein biosensor in a solution, the method comprising:
a) labeling a contractile protein with a first fluorescent probe and labeling a myosin binding protein-C (MyBP-C) with a second fluorescent probe to generate a fluorescent protein biosensor suitable for TR-FRET;
b) contacting the fluorescent protein biosensor with a drug candidate;
c) measuring FRET efficiency when structural changes in the fluorescent protein biosensor occur, wherein FRET efficiency is a proportion of donor molecules that have transferred excitation state energy to acceptor molecules; and
d) quantitating protein structural changes using the measured FRET efficiency.

15. The method of claim 14, wherein the contractile protein and the MyBP-C operably connect or bind in a physiological solution.

16. The method of claim 14, wherein the contractile protein comprises an actin protein, or a myosin protein, wherein the myosin protein comprises a full-length myosin protein or a fragment thereof, wherein the fragment of the myosin protein comprises a heavy meromyosin (HMM), a myosin subfragment 1 (S1), a myosin regulatory light chain (RLC) or a myosin subfragment 2 (S2); wherein the actin protein comprises an F-actin protein or a fragment thereof.

17. The method of claim 14, wherein the MyBP-C comprises a full-length MyBP-C or a fragment thereof, wherein the fragment of the MyBP-C comprises a C0-C2 fragment or a C0-C7 fragment.

18. The method of claim 14, wherein the first fluorescent probe comprises a fluorescent donor probe and the second fluorescent probe comprises a fluorescent acceptor probe, wherein the FRET efficiency is the proportion of donor molecules from the fluorescent donor probe that have transferred excitation state energy to acceptor molecules on the fluorescent acceptor probe.

19. The method of claim 14, wherein the MyBP-C further comprises one or more genetic modifications associated with hypertrophic cardiomyopathy and/or heart failure, wherein the one or more genetic modifications comprise E334K, L349R, and L352P.

20. The method of claim 14, wherein the first and second fluorescent probes are selected from a group consisting of IAEDANS, IAANS, CPM, IANBD, 5-IAF, TMP, ATTO FMAL, Alexa Fluor 488, Alexa Fluor 532, and Alexa Fluor 568.
